(19) United States
(12) Patent Application Publication
(10) Pub. No.: US 2026/0069585 A1
(43) Pub. Date: Mar. 12, 2026

(54) ISOQUINOLINE COMPOUNDS AND THEIR USE IN TREATING AhR MEDIATED DISEASES

(71) Applicant: Dermavant Sciences GmbH, Basel (CH)

(72) Inventors: David Scott RUBENSTEIN, Chapel Hill, NC (US); Kimberly Ann MCHALE, Durham, NC (US)

(73) Assignee: Dermavant Sciences GmbH, Basel (CH)

(21) Appl. No.: 19/107,415

(22) PCT Filed: Aug. 30, 2023
(86) PCT No.: PCT/US2023/073187

§ 371 Date: Feb. 27, 2025

Related U.S. Application Data
(60) Provisional application No. 63/373,929, filed on Aug. 30, 2022.

(51) Int. Cl. A61K 31/472; A61K 9/06; A61K 47/10; A61K 47/12; A61K 47/14; A61K 47/18; A61K 47/32; A61K 47/36

(52) U.S. Cl. CPC A61K 31/472; A61K 9/06; A61K 47/10; A61K 47/12; A61K 47/14; A61K 47/18; A61K 47/32; A61K 47/36

(57) ABSTRACT

The present invention is directed to methods of treating certain diseases comprising administering compounds or pharmaceutical compositions of Formula (I), Formula (Ia), or a pharmaceutically acceptable salt, solvate or hydrate thereof. Pharmaceutical compositions comprising a compound of Formula (I), Formula (Ia), or a pharmaceutically acceptable salt, solvate or hydrate thereof, are also described.

### CROSS-REFERENCE TO RELATED APPLICATIONS

This application claims priority to and benefit of U.S. Provisional Application No. 63/373,929, filed Aug. 30, 2022, the entire contents of which are hereby incorporated by reference.

### SUMMARY OF THE INVENTION

Various embodiments provide methods for the treatment and prevention of conditions associated with AhR imbalance, AhR mediated diseases and inflammatory disorders comprising administering compounds and compositions described herein. Such compounds described herein bind and activates the Aryl hydrocarbon Receptor (AhR), providing novel methods for the treatment of inflammatory disease states.

Some embodiments disclosed herein are directed to methods of treating a disease in a subject in need thereof comprising administering to the subject a compound of Formula (I) or a salt, solvate or hydrate thereof

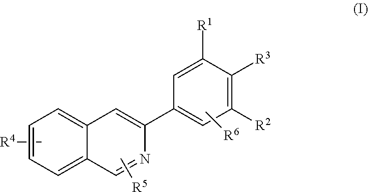

wherein
each of R¹ and R² is independently selected from the group consisting of OH, OR⁷, and H, provided that at least one of R¹ and R² is —OH or —OR⁷;
R⁷ is independently selected from the group consisting of optionally substituted C₁₋₆ alkyl, optionally substituted C₃₋₆ cycloalkyl, optionally substituted aryl, aryl C₁₋₆ alkyl and acyl;
R³ is selected from the group consisting of optionally substituted C₁₋₆ alkyl, optionally substituted C₂₋₆ alkenyl, optionally substituted C₂₋₆ alkynyl, optionally substituted aryl, optionally substituted aryl C₁₋₆ alkyl, optionally substituted C₃₋₆ cycloalkyl, optionally substituted C₄₋₆ cycloalkenyl, halo, cyano, —C(O)OR⁸, —NR⁹R¹⁰, —S(O)₂NR⁹R¹⁰, —C(O)R¹¹, —OR¹², —S(O)ₙ R¹³, and optionally substituted heterocyclyl;
R⁸ is selected from the group consisting of H, optionally substituted C₁₋₆ alkyl, optionally substituted C₃₋₆ cycloalkyl, optionally substituted aryl, and optionally substituted aryl C₁₋₆ alkyl;
each of R⁹ and R¹⁰ is independently selected from the group consisting of H, optionally substituted C₁₋₆ alkyl, optionally substituted C₃₋₆ cycloalkyl, optionally substituted aryl, and optionally substituted aryl C₁₋₆ alkyl, or alternatively, R⁹ and R¹⁰ together with the nitrogen atom to which they are attached form a 5-7 membered cyclic saturated or unsaturated ring;
R¹¹ is independently selected from the group consisting of H, optionally substituted C₁₋₆ alkyl, optionally substituted C₂₋₆ alkenyl, optionally substituted C₂₋₆ alkynyl, optionally substituted aryl, optionally substituted aryl C₁₋₆ alkyl, optionally substituted C₃₋₆ cycloalkyl, —NR⁹R¹⁰, and —OR¹²;
each of R¹² and R¹³ is independently selected from the group consisting of H, optionally substituted alkyl, optionally substituted alkenyl, optionally substituted alkynyl, optionally substituted aryl, optionally substituted aryl C₁₋₆ alkyl, and optionally substituted C₃₋₆ cycloalkyl;
R⁶ is selected from the group consisting of H, halo, hydroxyl, alkoxy, optionally substituted C₁₋₆ alkyl, halogenated alkyl; optionally substituted C₂₋₆ alkenyl, optionally substituted C₂₋₆ alkynyl, and optionally substituted aryl C₁₋₆ alkyl;
n is an integer having a value of 0, 1 or 2;
s is an integer having a value of 0, 1 or 2;
t is an integer having a value of 0 to 6;
R⁵ is selected from the group consisting of H, halo, optionally substituted C₁₋₆ alkyl, —C(O)OR¹⁴, —C(O)NR¹⁵R¹⁶, aryl and —C₁₋₆ alkylaryl;
R¹⁴ is selected from the group consisting of H, optionally substituted C₁₋₆ alkyl, optionally substituted C₃₋₆ cycloalkyl, optionally substituted aryl, and optionally substituted aryl C₁₋₆ alkyl;
each of R¹⁵ and R¹⁶ is independently selected from the group consisting of H, optionally substituted C₁₋₆ alkyl, optionally substituted C₃₋₆ cycloalkyl, optionally substituted aryl, optionally substituted aryl C₁₋₆ alkyl, and optionally substituted C₃₋₆ cycloalkyl; alternatively R¹⁵ and R¹⁶ together with the nitrogen to which they are attached, form a 5-7 membered cyclic saturated or unsaturated ring;
R⁴ is selected from the group consisting of H, halo, cyano, optionally substituted C₁₋₆ alkyl, optionally substituted C₂₋₆ alkenyl, optionally substituted C₂₋₆ alkynyl, optionally substituted aryl, optionally substituted aryl C₁₋₆ alkyl, optionally substituted C₃₋₆ cycloalkyl, —(CR¹⁸R¹⁹)ₜCOOR⁸, —(CR¹⁸R¹⁹)ₜOC(O)R⁸, —(CR¹⁸R¹⁹)ₜNR⁹R¹⁰, —(CR¹⁸R¹⁹)ₜC(O)NR⁹R¹⁰, —(CR¹⁸R¹⁹)ₜNR⁹C(O)R⁸, —(CR¹⁸R¹⁹)ₜS(O)₂NR⁹R¹⁰, —(CR¹⁸R¹⁹)ₜCOR¹¹, —(CR¹⁸R¹⁹)ₜCH(O), —(CR¹⁸R¹⁹)ₜOR¹², —(CR¹⁸R¹⁹)ₜS(O)ₛR¹³, optionally substituted heterocyclic, and optionally substituted heterocyclic C₁₋₆ alkyl; and
each of R¹⁸ and R¹⁹ is independently selected from the group consisting of H, optionally substituted C₁₋₆ alkyl, optionally substituted C₃₋₆ cycloalkyl, optionally substituted aryl, and optionally substituted aryl C₁₋₆ alkyl.

In some embodiments, the compound administered is selected from the group consisting of

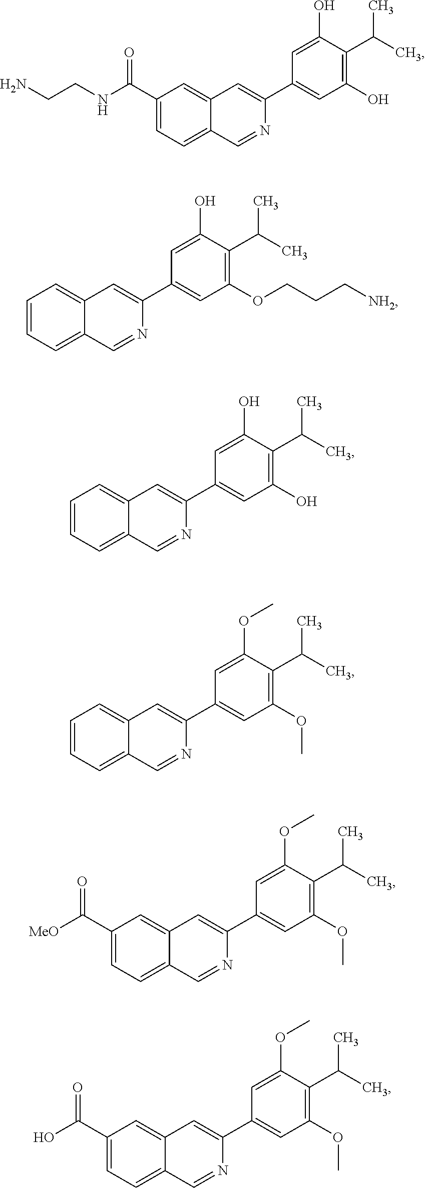 [SMILES: CC(C)c1c(O)cc(-c2cc3cc(C(=O)NCCN)ccc3cn2)cc1O ; CC(C)c1c(O)cc(-c2cc3ccccc3cn2)cc1OCCCN ; CC(C)c1c(O)cc(-c2cc3ccccc3cn2)cc1O ; COc1cc(-c2cc3ccccc3cn2)cc(OC)c1C(C)C ; COC(=O)c1ccc2cnc(-c3cc(OC)c(C(C)C)c(OC)c3)cc2c1 ; COc1cc(-c2cc3cc(C(=O)O)ccc3cn2)cc(OC)c1C(C)C]

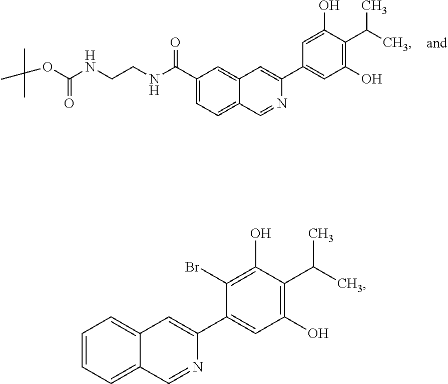 [SMILES: CC(C)c1c(O)cc(-c2cc3cc(C(=O)NCCNC(=O)OC(C)(C)C)ccc3cn2)cc1O ; CC(C)c1c(O)cc(-c2cc3ccccc3cn2)c(Br)c1O]

or a salt, solvate or hydrate thereof.

In some embodiments, the compound administered is of the formula

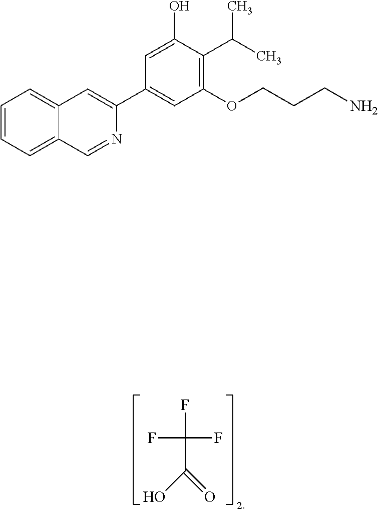

In some embodiments, the compound administered is of the formula

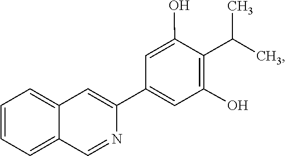 [SMILES: CC(C)c1c(O)cc(-c2cc3ccccc3cn2)cc1O]

or a pharmaceutically acceptable salt, solvate or hydrate thereof.

In some embodiments, the compound administered is 2-isopropyl-5-(isoquinolin-3-yl)benzene-1,3-diol, or a pharmaceutically acceptable salt solvate or hydrate thereof.

Some embodiments disclosed herein are directed to methods of treating a disease in a subject in need thereof comprising administering to the subject a pharmaceutical composition comprising: a compound according to any embodiment described herein, or a pharmaceutically acceptable salt, solvate or hydrate thereof, and a pharmaceutically acceptable carrier or diluent.

Some embodiments describe a method of treating or preventing a condition in a mammal associated with AhR imbalance, comprising administering to the mammal a therapeutically effective amount of any compound or pharmaceutical composition described herein.

Some embodiments describe a method of treating or preventing a disease in a subject, comprising administering to the subject a therapeutically effective amount of any compound or pharmaceutical composition described herein. In some embodiments, the compound or pharmaceutical composition treats such disease through enhancing regulatory B-cell activity to suppress inflammation. In some embodiments, the compound or pharmaceutical composition treats such disease through inhibiting activation and migration of polymorphonuclear leukocytes. In some embodiments, the compound or pharmaceutical composition treats such disease through AHR signaling in dendritic cells/antigen presenting cells and T cells and inhibiting activation of monocyte-derived dentritic cells/agentigen presentating cells, including production of inflammatory cytokins and upregulation of co-stimulatory molecules. In some embodiments, the compound or pharmaceutical composition treats such disease through inhibition of mast cell activation. In some embodiments, the compound or pharmaceutical composition treats such disease through suprresion or inhibition of angiogenesis. In some embodiments, the compound or pharmaceutical composition treats such disease through inhibition of ILC3 dysregulation, ILC2 dysregulation and promote antitumor action in ILC and NK cells. In some embodiments, the compound or pharmaceutical composition treats such disease through modulating macrophages and phagocytosis of apoptotic cells. In some embodiments, the compound or pharmaceutical composition treats such disease through regulating epithelial cells (including skin, gut, and/or respiratory epithelial cells), and/or through interacting with or affecting signaling between such epithelial cells and immune cells. In some embodiments, the compound or pharmaceutical composition treats such disease through suppression of antigen-presenting cell activity. In some embodiments, the compound or pharmaceutical composition treats such disease through affecting B-cell and plasma cell activity, thereby suppressing autoantibody and/or pathogenic antibody production.

Some embodiments describe a method of treating or preventing a disease in a subject, comprising administering to the subject a therapeutically effective amount of any compound or pharmaceutical composition described herein. In some embodiments, the disease is selected from the group consisting of Pemphigus foliaceus, Pemphigus vulgaris, paraneoplastic pemphigus, bullous pemphigoid, epidermolysis bullosa acquisita, lupus erythematosus, dermatomyositis, myasthenia gravis, goodpadture's syndrome, and autoimmune thyroiditis. In some embodiments, the disease is selected from the group consisting of Sweet's syndrome, pustular psoriasis, pyoderma gangrenosum, hidradenitis suppuritiva, leukocytoclastic vasculitis Wegener's Granulomatosis and neutrophilic dermatoses. In some embodiments, the disease is selected from the group consisting of allergic diseases, autoimmune diseases, inflammatory airway disease, allergic rhinitis, autoimmune blistering diseases including Pemphigus vulgaris and Pemphigus foliaceus, bullous pemphigoid, inflammatory joint disease, Schamberg's disease or other pigmented purpuric dermatoses (PPDs), lupus vulgaris, and dermatomyositis. In some embodiments, the disease is selected from the group consisting of mastocytosis, urticaria, rosacea, fibromyalgia, chronic pain syndromes, chronic pruritis, chronic itch, and allergic rhinitis. In some embodiments, the disease is selected from the group consisting of arteriosclerosis, benign tumor growth and malignant tumor growth. In some embodiments, the disease is selected from the group consisting of inflammatory airway disease, allergic rhinitis, Schamberg's disease or other pigmented purpuric dermatoses (PPDs), and inflammatory joint disease. In some embodiments, the disease is vitiligo. In some embodiments, the disease is an oral disease selected from the group consisting of gingivitis (including gingivitis having a TH17-driven component), mucositis, apthous stomatitis, lichen planus, and erosive oral lichen planus. In some embodiments, the disease is an ophthalmic disease selected from the group consisting of blepharitis, dry eye disease, meibomian gland dysfunction, keratoconjunctivitis, atopic keratoconjunctivitis, vernal keratoconjunctivitis, conjunctivitis, uveitis, and age-related macular degeneration. In some embodiments, the compound is 2-isopropyl-5-(isoquinolin-3-yl)benzene-1,3-diol, or a pharmaceutically acceptable salt, solvate or hydrate thereof.

### DETAILED DESCRIPTION OF THE INVENTION

The aryl hydrocarbon receptor (AhR), a member of the bHLH-PAS family of transcription factors, is a cytosolic ligand-activated transcription factor that senses diverse endogenous and exogenous molecules mediating multiple biological activities It is best known for mediating the toxic effects of environmental contaminants such as TCDD (dioxin) and a range of other xenobiotic substances. Recent evidence points to AhR as a highly-conserved pathway that can modulate inflammatory responses, thus the AhR pathway could be an important target for treating inflammatory diseases.

There is a wide expression of AhR in several cell types of the skin, including keratinocytes, fibroblasts, melanocytes, and skin immune cells. In keratinocytes, AhR signaling controls the expression of epidermal differentiation genes, such as filaggrin, loricrin, and homerin, thus promoting skin barrier formation. Additionally, it has been shown that AhR plays a critical role as a regulator of both innate and adaptive immune responses by impacting the balance of Th17 and Treg T cells. Th17-associated cytokines, including IL-17, contribute to the immunopathogenesis of inflammatory skin diseases such as psoriasis. Therefore, attention has recently been drawn to AhR as a target for the treatment or prevention of inflammatory skin diseases, concurrently highlighting the need for better topical treatments.

Compounds such as those described herein, that bind and activate the Aryl hydrocarbon Receptor (AhR), provide for a novel class of anti-inflammatory compounds with AhR-dependent cytokine modulation useful for the treatment of the disease states described herein.

Thus, the beneficial effects of AhR activation provide for new therapeutic interventions in the treatment of certain disease states. The need exists for better topical treatment of skin diseases, and in particular chronic inflammatory skin diseases. Suitably, a compound which binds and activate the Aryl hydrocarbon Receptor (AhR) in multiple cell types, including cells of the human skin, will provide a novel and useful treatment for inflammatory disease states. The present invention thus provides for a novel class of anti-inflammatory compounds with AhR-dependent cytokine modulation for treatment thereof.

### Definitions

It should be understood that the terms “a” and “an” as used herein refer to “one or more” of the recited components. It will be clear to one of ordinary skill in the art that the use of the singular includes the plural unless specifically stated otherwise.

The term “about” means within an acceptable range for the particular parameter specified as determined by one of ordinary skill in the art, which will depend, in part, on how the value is measured or determined, i.e., the limitations of the measurement system. For example, “about” can mean a range of 10% of a given value. For example, about 55% means 45% to 55%.

As used herein, “acyl” means an alkyl or aryl group bonded through a carbonyl group —C(O)—. For example, acyl includes a C₁₋₆ alkanoyl. Typical acyl groups include acetyl, benzoyl, and the like.

The terms “administering” and “administration” are used herein to mean any method which in sound medical practice delivers the compound or pharmaceutical composition thereof to a subject in such a manner as to provide the desired therapeutic effect. In some embodiments the compound is in a pharmaceutical emulsion composition.

As used herein, the term “alkoxy” refers to an —O-alkyl group containing a specified number of carbon atoms. For example, C₁₋₆ alkoxy means alkoxy group containing at least 1, and at most 6, carbon atoms. Examples of “alkoxy” as used herein include, but are not limited to, methoxy, ethoxy, propoxy, prop-2-oxy, butoxy, but-2-oxy, 2-methylprop-1-oxy, 2-methylprop-2-oxy, pentoxy or hexyloxy.

As used herein, “alkyl” refers to a monovalent saturated hydrocarbon chain having the specified number of carbon member atoms. For example, C₁₋₆ alkyl refers to an alkyl group having from 1 to 6 carbon member atoms. Alkyl groups may be straight or branched. Representative branched alkyl groups have one, two, or three branches. Alkyl includes methyl, ethyl, propyl, (n-propyl and isopropyl), butyl (n-butyl, isobutyl, s-butyl, and t-butyl), and n-pentyl, and the like.

The term “alkylene” means a linker which is a straight or branched carbon chain having 1 to 6 carbon atoms and having two bonding sites. Examples of C₁₋₆ alkylene linker groups include but are not limited to —CH₂—, —CH₂CH₂—, —CH₂CH₂CH₂CH₂—, —CH₂CH(CH₃)CH₂—, and the like.

The term “and/or” as used herein covers both additively and also alternatively the individual elements of a list which are thus linked so that these elements are to be understood as linked selectively with “and” or respectively with “or”. Furthermore, the terms used in the singular of course also comprise the plural.

The term “applying” as used herein refers to any method which, in sound medical or cosmetic practice, delivers a topical composition to the subject in such a manner so as to provide a positive effect on a dermatological disorder, condition, or appearance.

As used herein, the term “aryl” refers to substituted or unsubstituted hydrocarbon aromatic rings such as phenyl, naphthyl and the like.

As used herein, the term “arylalkyl” or “araalkyl” refers to an aryl ring such as benzene or naphthalene and a connecting C₁₋₆ alkyl moiety, unless otherwise indicated, such as

—(CH₂)ₙphenyl wherein n is 1-6.

As used herein, the terms “compound(s) of the invention” or “compound(s) of this invention” mean a compound, as defined herein, in any form, i.e., any salt or non-salt form (e.g., as a free acid or base form, or as a salt, particularly a pharmaceutically acceptable salt thereof) and any physical form thereof (e.g., including non-solid forms (e.g., liquid or semi-solid forms), and solid forms (e.g., amorphous or crystalline forms, specific polymorphic forms, solvate forms, including hydrate forms (e.g., mono-, di- and hemi-hydrates)), and mixtures of various forms.

Throughout the application, descriptions of various embodiments use “comprising” language, however in some specific instances, an embodiment can alternatively be described using the language “consisting essentially of” or “consisting of.”

The term “pharmaceutically acceptable excipient or diluent” as used herein refers to any inactive ingredient present in a composition for use in a pharmaceutical composition described herein.

“Effective amount”, “pharmaceutically effective amount” or “therapeutically effective amount” is used herein to refer to an amount of the active ingredient sufficient to have a therapeutic effect upon administration, e.g., that amount which will cause an improvement or change in the condition for which it is administered. Effective amounts will vary with the particular condition being treated, the severity of the condition, the duration of the treatment, the stage of advancement of the condition, the body surface area affected with the clinical condition (for topical administration), and the specific components of the composition. The amount is sufficient to treat a disorder, disease or condition or one or more of its symptoms and/or to prevent the occurrence of the disease or disorder, and can be determined by standard clinical techniques. Appropriate amounts in any given instance will be readily apparent to those skilled in the art or capable of determination by routine experimentation. In some embodiments, compositions of the invention are generally applied in a topical manner to the affected area, i.e., localized application to the skin region where the clinical abnormality is manifest.

As used herein, the term “haloalkyl” or “halo substituted alkyl” refers to a straight or branched saturated hydrocarbon chain containing a specified number of carbon atoms, substituted with halo atoms. For example, halo C₁₋₆ alkyl means a straight or branched alkyl group containing at least 1, and at most 6, carbon atoms, substituted with 1 to 3 halo atoms per carbon atom. Examples of “haloalkyl” as used herein include, but are not limited to, fluoromethyl, difluoromethyl, and trifluoromethyl.

As used herein, the terms “halogen” and “halo” include fluorine, chlorine, bromine and iodine, and fluoro, chloro, bromo, and iodo, respectively.

As used herein, the terms “heteroaryl ring”, “heteroaryl moiety”, and “heteroaryl” mean a monocyclic five to seven membered unsaturated hydrocarbon ring containing at least one heteroatom selected from oxygen, nitrogen and sulfur. Examples of heteroaryl rings include, but are not limited to, furyl, pyranyl, thienyl, pyrrolyl, oxazolyl, thiazolyl, isoxazolyl, isothiazolyl, imidazolyl, pyrazolyl, oxadiazolyl, oxathiadiazolyl, triazolyl, tetrazolyl, thiadiazolyl, pyridyl, pyridazinyl, pyrimidinyl, pyrazinyl, triazinyl, and uracil. The terms “heteroaryl ring”, “heteroaryl moiety”, and “heteroaryl” as used herein, also refer to fused aromatic rings comprising at least one heteroatom selected from oxygen, nitrogen and sulfur. Each of the fused rings may contain five or six ring atoms. Examples of fused aromatic rings include, but are not limited to, indolyl, isoindolyl, indazolyl, indolizinyl, azaindolyl, benzoxazolyl, benzimidazolyl, benzothiazolyl, benzofuranyl, benzothiophenyl, quinolyl, isoquinolyl, quinazolinyl, quinoxalinyl, naphthyridinyl, cinnolinyl, purinyl, and phthalazinyl.

As used herein, the term “heteroarylalkyl” means a C₁₋₆ alkyl as defined above, (unless otherwise defined) attached to a heteroaryl moiety as also defined herein unless otherwise indicated.

As used herein, the term “heterocyclicalkyl” or “heterocyclylalkyl” means a C₁₋₆ alkyl as defined above, (unless otherwise defined) attached to a heterocyclic moiety as also defined herein unless otherwise indicated.

As used herein, the term “heterocyclic” or “heterocyclyl” (on its own or in any combination, such as “heterocyclylalkyl”) is used herein to mean a saturated or partially unsaturated 4 to 10 membered ring system in which one or more rings contain one or more heteroatoms selected from the group consisting of N, O, S, or S(O)q, wherein q is 0 or an integer having a value of 1 or 2. Examples include, but not limited to, tetrahydropyrrolyl, tetrahydropyranyl, tetrahydrofuranyl, tetrahydrothiophenyl (including oxidized versions of the sulfur moiety), pyrrolidinyl, piperidinyl, piperazinyl, morpholinyl, thiomorpholinyl (including oxidized versions of the sulfur moiety), or imidazolidinyl.

As used herein in the in vitro skin penetration studies, the term “epidermis” includes the stratum corneum and tissue or layers down to the basement membrane, as isolated by heat separation treatment.

As used herein in the in vitro skin penetration studies with ex vivo human abdominal skin dermatomed at a thickness of 500 microns (+/−100 microns) or 750 (+/−100 microns), the term “epidermis” is the top/superficial layer obtained after a washing/tape striping procedure followed by heat separation, and the term “dermis” is the underlying layer.

As used herein, the term “independently” means that where more than one substituent is selected from a number of possible substituents, those substituents may be the same or different. That is, each substituent is separately selected from the entire group of recited possible substituents.

The terms “modulate” or “modulates” refer to an increase or decrease in the amount, quality, or effect of a particular activity.

As used herein, the term “oxo” represents a double-bonded oxygen moiety; for example, if attached directly to a carbon atom it forms a carbonyl moiety (C═O).

As used herein, the term “hydroxy” or “hydroxyl” is intended to mean the radical —OH.

As used herein, the term “sulfinyl” is used herein to mean the oxide S(O) of the corresponding sulfide, the term “thio” refers to the sulfide, and the term “sulfonyl” refers to the fully oxidized S(O)₂ moiety.

As used herein, the term “optionally” means that the subsequently described event(s) may or may not occur, and includes both event(s) that occur and event(s) that do not occur.

As used herein, “optionally substituted”, unless specifically defined herein shall mean the moiety may be optionally substituted one or more times, such as one to three times, independently with halo, e.g. fluoro, chloro, bromo or iodo; hydroxy; hydroxy substituted C₁₋₃ alkyl; C₁₋₃ alkoxy, such as methoxy or ethoxy; halo substituted C₁₋₃ alkoxy; S(O)ₘC₁₋₃ alkyl, such as methyl thio, methylsulfinyl or methyl sulfonyl; NR²²R²³, wherein R²² and R²³ are independently selected from H or C₁₋₃ alkyl or wherein R²² and R²³ together with the nitrogen to which they are attached form a 5 to 7 membered ring which optionally contains an additional heteroatom selected from O, N, or S; C ₁₋₃ alkyl; C₃₋₇ cycloalkyl, or C₃₋₇ cycloalkyl C₁₋₃ alkyl group, such as cyclopropyl methyl; halosubstituted C₁₋₃ alkyl, such CF₂CF₂H, or CF₃; optionally substituted aryl, such as phenyl or optionally substituted aryl C₁₋₃ alkyl, such as benzyl or phenethyl, and wherein these aryl containing moieties may also be substituted one to two times by halo; hydroxy; hydroxy substituted C₁₋₃ alkyl; C₁₋₃ alkoxy; S(O)ₘC₁₋₃ alkyl; amino, mono and di-substituted C₁₋₃ alkylamino; C₁₋₃ alkyl, or CF₃. In addition, it will be appreciated by those skilled in the art that the compounds of this invention, depending on further substitution, may exist in other tautomeric forms. All tautomeric forms of the compounds described herein are intended to be encompassed within the scope of the present invention. It is to be understood that any reference to a named compound of this invention is intended to encompass all tautomers of the named compound and any mixtures of tautomers of the named compound.

As used herein, “patients” or “subjects” (used interchangeabley) includes human patients, including adult, teens and children (e.g., pediatric patients). A pediatric patient can include teenagers under the age of 18. A child for purposes herein is under the age of 12.

As used herein, “pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio. The terms “pharmaceutically acceptable” and “dermatologically acceptable” mean approvable by a regulatory agency or listed in a Pharmacopeia or other generally recognized guide for use in animals, and more particularly in humans.

The term “pharmaceutically acceptable salt thereof” refers to salts that are safe and effective for use in the patient and possess the desired pharmaceutical activity. Salts encompassed within the term pharmaceutically acceptable salts refer to non-toxic salts of the compounds of this invention. Such salts include compounds wherein the parent compound is modified by making acid or base salts thereof.

As used herein, the term “skin penetration” refers to the diffusion of a compound, preferably a compound of Formula (I) or a pharmaceutically acceptable salt thereof through the stratum corneum and into the epidermis and/or dermis of the skin.

“Substantially free” of a specified component refers to a composition with less than about 1% by weight of the specified component. “Free” of a specified component refers to a composition where the specified component is absent.

As used herein, the term “substituted” in reference to a group indicates that one or more hydrogen atom attached to a member atom within the group is replaced with a substituent selected from the group of defined substituents. It should be understood that the term “substituted” includes the implicit provision that such substitution be in accordance with the permitted valence of the substituted atom and the substituent and that the substitution results in a stable compound (i.e., one that does not spontaneously undergo transformation such as by rearrangement, cyclization, or elimination and that is sufficiently robust to survive isolation from a reaction mixture). When it is stated that a group may contain one or more substituents, one or more (as appropriate) member atoms within the group may be substituted. In addition, a single member atom within the group may be substituted with more than one substituent as long as such substitution is in accordance with the permitted valence of the atom. Suitable substituents are defined herein for each substituted or optionally substituted group.

The term “topical” delivery or “topical” administration refers to application of a drug-containing formulation to the skin to directly treat cutaneous disorders or the cutaneous manifestations of a disease with the intent of substantially directing the pharmacological effect of the drug to the surface of the skin or within the skin. The term “topical” delivery also includes dermal, inhaled and ocular/otic administration. “Topical” administration also refers to application to and diffusion through the stratum corneum, including but not limited to application to psoriatic lesions and broken skin.

The term “treating” or “treatment” as used herein refers to administration of a compound or agent to a subject who has a disorder or is at risk of developing the disorder with the purpose to cure, alleviate, relieve, remedy, delay the onset of, prevent, or ameliorate the disorder, the symptom of the disorder, the disease state secondary to the disorder, or the predisposition toward the disorder. Treatment need not mean that the condition or disorder is totally cured. A useful pharmaceutical composition, e.g., a pharmaceutical emulsion composition, herein need only to reduce the severity of the condition or disorder, reduce the severity of symptoms associated therewith, provide improvement to a patient's quality of life, or delay, prevent or inhibit the onset of the condition or disorder. A treatment need not be effective in every member of a population, e.g., a population of patients with atopic dermatitis, to have clinical utility, as is recognized in the medical and pharmaceutical arts.

Concentrations, amounts, solubilities, and other numerical data may be presented herein in a range format. It is to be understood that such range format is used merely for convenience and brevity and should be interpreted flexibly to include not only the numerical values explicitly recited as the limit of the range, but also to include all the individual numerical values or sub-ranges encompassed within that range as if each numerical value and sub-range is explicitly recited. All numbers expressing quantities, percentages or proportions, and other numerical values used in the specification, are to be understood as being modified in all instances by the term “about.”

For example, a concentration range of 0.1 to 5 ng/ml should be interpreted to include not only the explicitly recited concentration limits of 0.1 ng/ml and 5 ng/ml but also to include individual concentrations such as 0.2 ng/ml, 0.8 ng/ml, 1.0 ng/ml 2.2 ng/ml, 3.6 ng/mol, and sub-ranges such as 0.3-2.5 ng/ml, 1.8-3.2 ng/ml, etc. This interpretation should apply regardless of the breadth of the range or the characteristic being described.

Any concentration range, percentage range or ratio range recited herein is to be understood to include concentrations, percentages or ratios of any integer within that range and fractions thereof, such as one tenth and one hundredth of an integer, unless otherwise indicated.

Other terms used herein are intended to be defined by their well-known meanings in the art.

The alternative definitions for the various groups and substituent groups of Formula (I) and Formula (Ia) provided throughout the specification are intended to particularly describe each compound species disclosed herein, individually, as well as groups of one or more compound species. The scope of this invention includes any combination of these group and substituent group definitions.

### Compounds Administered in Method Embodiments Described Herein

In some embodiments, the present disclosure describes administering a compound of Formula (I):

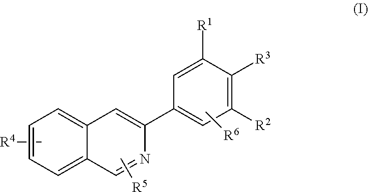

or a salt, solvate or hydrate thereof.

Each of substituents R¹ and R² of Formula (I) is independently selected from the group consisting of OH, OR⁷, and H, provided that at least one of R¹ and R² is OH or OR⁷.

Substituent R⁷ of Formula (I) is independently selected from the group consisting of optionally substituted C₁₋₆ alkyl, optionally substituted C₃₋₆ cycloalkyl, optionally substituted aryl, optionally substituted aryl C₁₋₆ alkyl and acyl.

Substituent R³ of Formula (I) is selected from the group consisting of optionally substituted C₁₋₆ alkyl, optionally substituted C₂₋₆ alkenyl, optionally substituted C₂₋₆ alkynyl, optionally substituted aryl, optionally substituted aryl C₁₋₆ alkyl, optionally substituted C₃₋₆ cycloalkyl, optionally substituted C₄₋₆ cycloalkenyl, halo, cyano, —C(O)OR⁸, —NR⁹R¹⁰, —S(O)₂NR⁹R¹⁰, —C(O)R¹¹, —OR¹², —S(O)ₙR¹³, and optionally substituted heterocyclic ring.

The substituent R⁸ of Formula (I) is independently selected from the group consisting of H, optionally substituted C₁₋₆ alkyl, optionally substituted C₃₋₆ cycloalkyl, optionally substituted aryl, and optionally substituted aryl C₁₋₆ alkyl;

Each of R⁹ and R¹⁰ of Formula (I) is independently selected from the group consisting of H, optionally substituted C₁₋₆ alkyl, optionally substituted C₃₋₆ cycloalkyl, optionally substituted aryl, and optionally substituted aryl C₁₋₆ alkyl. Alternatively, substituents R⁹ and R¹⁰ together with the nitrogen atom to which they are attached, form a 5-7 membered cyclic saturated or unsaturated ring.

Substituent R¹¹ of Formula (I) is independently selected from the group consisting of hydrogen, optionally substituted C₁₋₆ alkyl, optionally substituted C₂₋₆ alkenyl, optionally substituted C₂₋₆ alkynyl, optionally substituted aryl, optionally substituted aryl C₁₋₆ alkyl, optionally substituted C₃₋₆ cycloalkyl, —NR⁹R¹⁰, and —OR¹².

Each of substituents R¹² and R¹³ of Formula (I) is independently selected from the group consisting of H, optionally substituted alkyl, optionally substituted alkenyl, optionally substituted alkynyl, optionally substituted aryl, optionally substituted aryl C₁₋₆ alkyl, and optionally substituted C₃₋₆ cycloalkyl.

Substituent R⁶ of Formula (I) is selected from the group consisting of H, halo, hydroxyl, alkoxy, optionally substituted C₁₋₆ alkyl, halogenated alkyl, optionally substituted C₂₋₆ alkenyl, optionally substituted C₂₋₆ alkynyl, and optionally substituted aryl C₁₋₆ alkyl.

Subscript n of Formula (I) is an integer having a value of 0, 1 or 2.

Subscript s of Formula (I) is an integer having a value of 0, 1 or 2.

Subscript t of Formula (I) is an integer having a value of 0 to 6.

The substituent R⁵ of Formula (I) is selected from the group consisting of H, halo, optionally substituted C₁₋₆ alkyl, —C(O)OR¹⁴, —C(O)NR¹⁵R¹⁶, optionally substituted aryl, and optionally substituted —C₁₋₆ alkylaryl.

Substituent R¹⁴ of Formula (I) is selected from the group consisting of H, optionally substituted C₁₋₆ alkyl, optionally substituted C₃₋₆ cycloalkyl, optionally substituted aryl, and optionally substituted aryl C₁₋₆ alkyl.

Each of substituents R¹⁵ and R¹⁶ of Formula (I) is independently selected from the group consisting of H, optionally substituted C₁₋₆ alkyl, optionally substituted C₃₋₆ cycloalkyl, optionally substituted aryl, optionally substituted aryl C₁₋₆ alkyl, and optionally substituted C₃₋₆ cycloalkyl. Alternatively, R¹⁵ and R¹⁶ together with the nitrogen to which they are attached, forms a 5-7 membered cyclic saturated or unsaturated ring.

Substituent R⁴ of Formula (I) is selected from the group consisting of H, halo, cyano, optionally substituted C₁₋₆ alkyl, optionally substituted C₂₋₆ alkenyl, optionally substituted C₂₋₆ alkynyl, optionally substituted aryl, optionally substituted aryl C₁₋₆ alkyl, optionally substituted C₃₋₆ cycloalkyl, —(CR¹⁸R¹⁹)ₜCOOR⁸, —(CR¹⁸R¹⁹)ₜ OC(O)R⁸; —(CR¹⁸R¹⁹)ₜNR⁹R¹⁰, —(CR¹⁸R¹⁹)ₜC(O)NR⁹R¹⁰, —(CR¹⁸R¹⁹)ₜNR⁹C(O)R⁸, —(CR¹⁸R¹⁹)ₜS(O)₂NR⁹R¹⁰, —(CR¹⁸R¹⁹)ₜCOR¹¹, —(CR¹⁸R¹⁹)ₜCH(O), —(CR¹⁸R¹⁹)ₜOR¹², —(CR¹⁸R¹⁹)ₜS(O)ₛR¹³; optionally substituted heterocyclic and optionally substituted heterocyclicC₁₋₆ alkyl.

Each of substituent R¹⁸ and R¹⁹ of Formula (I) is independently selected from the group consisting of H, optionally substituted C₁₋₆ alkyl, optionally substituted C₃₋₆ cycloalkyl, optionally substituted aryl, and optionally substituted aryl C₁₋₆ alkyl.

In some embodiments, the salt is a pharmaceutically acceptable salt.

In some embodiments each of R¹ and R² is independently OH. In some embodiments, one of R¹ and R² is OH and the other is H. In some embodiments, one of R¹ and R² is OH and the other is OR⁷ and in some embodiments both R¹ and R² are OR⁷ wherein each R⁷ is independently selected from the group consisting of optionally substituted C₁₋₆ alkyl, optionally substituted C₃₋₆ cycloalkyl, optionally substituted aryl, optionally substituted aryl C₁₋₆ alkyl and acyl. In some embodiments, R⁷ is optionally substituted C₁₋₄ alkyl. In some embodiments, R⁷ is methyl or ethyl. In other embodiments, R⁷ is optionally substituted C₁₋₄ alkyl substituted with NR²²R²³. In some embodiments, each of R²² and R²³ is independently selected from the group consisting of H and C₁₋₃ alkyl, such as —(CH₂)₃NH₂. In some embodiments each of R¹ and R² is methoxy. In some embodiments R¹ is OH and R² is OR⁷, wherein R⁷ is a substituted C₁₋₆ alkyl. In some embodiments R¹ is OH and R² is OR⁷, wherein R⁷ is C₁₋₆ alkyl substituted with NR²²R²³. In some embodiments R¹ is OH and R² is —O(CH₂)₃NH₂.

When R⁷ is an optionally substituted moiety, the moiety being substituted may be optionally substituted one or more times, such as one to three times, independently with halo; hydroxyl; hydroxy substituted —C₁₋₃ alkyl; C₁₋₃ alkoxy; halosubstituted C₁₋₃ alkoxy; —S(O)ₘC₁₋₃, wherein m is an integer having a value of 0, 1 or 2; —NR²²R²³, wherein R²² and R²³ are independently selected from H or a C₁₋₃ alkyl or wherein R²² and R²³ together with the nitrogen to which they are attached form a 5 to 7 membered ring which optionally contains an additional heteroatom selected from O, N, or S; C₃₋₇ cycloalkyl; halosubstituted C₁₋₃ alkyl, such as CF₂CF₂H, or CF₃; or optionally substituted aryl, wherein the aryl moiety may also be optionally substituted one to two times by halo; hydroxyl; hydroxy substituted —C₁₋₃ alkyl; C₁₋₃ alkoxy; —S(O)ₘC₁₋₃ alkyl, wherein m is an integer having a value of 0, 1 or 2; amino; mono and di-substituted C₁₋₃ alkylamino; C₁₋₃ alkyl; or CF₃.

In some embodiments R⁷ is optionally substituted C₁₋₆ alkyl. In some embodiments R⁷ is C₁₋₆ alkyl substituted with NR²²R²³. In some embodiments R⁷ is C₁₋₆ alkyl substituted with NH₂. In some embodiments R⁷ is —(CH₂)₃NH₂.

In some embodiments, when R³ is an optionally substituted moiety, the moiety may be substituted independently one or more times, such as one to three times. In some embodiments, the moieties may be optionally substituted independently one to three times with halo, hydroxy, C₁₋₃ alkoxy, C₁₋₃ alkyl, aryl or arylalkyl.

In some embodiments R³ is selected from the group consisting of optionally substituted C₃₋₆ alkyl and optionally substituted C₃₋₆ cycloalkyl. In some embodiments, the C₃₋₆ alkyl is isopropyl, n-propyl, n-butyl, t-butyl, sec-butyl, n-pentyl, isopentyl, 2-methylbutyl, n-hexyl, and the like. In some embodiments, the alkyl is isopropyl or t-butyl. In some embodiments, the C₃₋₆ alkyl is isopropyl. In some embodiments, the C₃₋₆ cycloalkyl is a cyclopropyl, cyclopentyl or cyclohexyl. In some embodiments, the C₃₋₆ cycloalkyl is cyclopentyl.

In some embodiments R³ is a heterocyclic ring.

In some embodiments R³ is isopropyl.

In some embodiments, R⁶ is selected from the group consisting of H, halo, hydroxyl, C₁₋₃ alkoxy, optionally substituted C₁₋₆ alkyl, halogenated alkyl, optionally substituted C₂₋₆ alkenyl, optionally substituted C₂₋₆ alkynyl, and optionally substituted aryl C₁₋₆ alkyl. In some embodiments R⁶ is selected from the group consisting of H, halo, hydroxyl, C₁₋₃ alkoxy, optionally substituted C₁₋₃ alkyl, and halogenated alkyl. In some embodiments, R⁶ is selected from the group consisting of H and halo. In some embodiments, R⁶ is selected from the group consisting of H and bromo. In some embodiments, R⁶ is H.

In some embodiments R⁵ is selected from the group consisting of H, halo, optionally substituted —C₁₋₆ alkyl, —C(O)OR¹⁴, —C(O)NR¹⁵R¹⁶, aryl and —C₁₋₆ alkylaryl. In some embodiments, R⁵ is selected from the group consisting of H, halo, optionally substituted C₁₋₆ alkyl, C(O)OR¹⁴, and C(O)NR¹⁵R¹⁶. In some embodiments, R⁵ is selected from the group consisting of H, halo, optionally substituted C₁₋₆ alkyl, and C(O)OR¹⁴. In some embodiment, R⁵ is selected from the group consisting of H and optionally substituted C₁₋₆ alkyl. In some embodiments, R⁵ is selected from the group consisting of H, C(O)OR¹⁴, and C(O)NR¹⁵R¹⁶. In some embodiments, R⁵ is optionally substituted C₁₋₃ alkyl. In some embodiments when R⁵ is an optionally substituted —C₁₋₆ alkyl moiety, the —C₁₋₆ alkyl is optionally substituted one to three times, independently with halo; hydroxyl; C₁₋₃ alkoxy; halosubstituted C₁₋₃ alkoxy; —S(O)ₘC₁₋₃ alkyl, wherein m is an integer having a value of 0, 1 or 2; —NR²⁰R²¹, wherein R²⁰ and R²¹ are independently selected from the group consisting of H and C₁₋₃ alkyl; halo substituted C₁₋₃ alkyl, such as CF₂CF₂H, or CF₃; or aryl. In some embodiments, R⁵ is H.

In some embodiments, R⁴ is selected from the group consisting of H, —(CR¹⁸R¹⁹)ₜCOOR⁸, —(CR¹⁸R¹⁹)ₜC(O)NR⁹R¹⁰, —(CR¹⁸R¹⁹)ₜNR⁹R¹⁰, optionally substituted C₁₋₆ alkyl, —(CR¹⁸R¹⁹)ₜOR¹² and —(CR¹⁸R¹⁹)ₜS(O)ₛR¹³. In some embodiments, R⁴ is selected from the group consisting of H, —(CR¹⁸R¹⁹)ₜCOOR⁸, and —(CR¹⁸R¹⁹)ₜC(O)NR⁹R¹⁰; wherein t is 0, R⁹ is H and R¹⁰ is optionally substituted C₁₋₆ alkyl. In some embodiments R⁴ is selected from the group consisting of H, —COOH, —COOCH₃ and —CONH(CH₂)₂NH₂. In some embodiments R⁴ is H.

In some embodiments, t is an integer having a value of 0, 1, 2, or 3. In some embodiments, t is 0.

In some embodiments, each of R⁹ and R¹⁰ is independently selected from the group consisting of H and optionally substituted C₁₋₆ alkyl.

In some embodiments, R¹¹ is selected from the group consisting of H, and optionally substituted C₁₋₆ alkyl.

In some embodiments, each of R¹² and R¹³ is independently selected from the group consisting of H and optionally substituted alkyl.

In some embodiments, n is 0 or 2. In some embodiments, n is 0. In some embodiments, n is 2.

In some embodiments, each of R¹⁸ and R¹⁹ is independently selected from the group consisting of H and optionally substituted C₁₋₆ alkyl.

Some embodiments describe a compound of Formula (I) wherein:
R¹ is selected from the group consisting of OH and OR⁷, wherein R⁷ is optionally substituted C₁₋₆ alkyl;
R² is selected from the group consisting of OH and OR⁷, wherein R⁷ is optionally substituted C₁₋₆ alkyl;
R³ is optionally substituted C₁₋₆ alkyl;
R⁴ is selected from the group consisting of H, —(CR¹⁸R¹⁹)ₜCOOR⁸, —(CR¹⁸R¹⁹)ₜC(O)NR⁹R¹⁰; wherein t is 0, R⁸ is selected from H and optionally substituted C₁₋₆ alkyl, R⁹ is H and R¹⁰ is optionally substituted C₁₋₆ alkyl;
R⁵ is H; and
R⁶ is H or halo.

Some embodiments describe a compound of Formula (I) wherein:
R¹ is selected from the group consisting of OH and OR⁷, wherein R⁷ is alkyl;
R² is selected from the group consisting of OH and OR⁷, wherein R⁷ is C₁₋₆ alkyl substituted with NR²²R²³; wherein R²² and R²³ are independently selected from the group consisting of H and C₁₋₃ alkyl;
R³ is optionally substituted C₁₋₆ alkyl;
R⁴ is selected from the group consisting of H, —COOH, —COOCH₃, and —C(O)NR⁹R¹⁰; wherein R⁹ is H; and R¹⁰ is selected from the group consisiting of amino substituted C₁₋₆ alkyl and —(CH₂)₂NHC(O)O-t-butyl;
R⁵ is H; and
R⁶ is selected from the group consisting of H and halo.

Some embodiments describe a compound of Formula (I) wherein:
R¹ is selected from the group consisting of OH and —OCH₃;
R² is selected from the group consisting of OH. —OCH₃ and —O—(CH₂)₃NH₂;
R³ is C₁₋₆ alkyl;
R⁴ is selected from the group consisting of H, —COOH, —COOCH₃, —C(O)NH(CH₂)₂NH₂; and —C(O)NH(CH₂)₂NHC(O)O-t-butyl;
R⁵ is H; and
R⁶ is selected from the group consisting of H and bromo.

Some embodiments describe a compound of Formula (I) wherein:
R¹ is selected from the group consisting of OH and —OCH₃;
R² is selected from the group consisting of OH, —OCH₃ and —O—(CH₂)₃NH₂;
R³ is isopropyl;
R⁴ is selected from the group consisting of H, —COOH, —COOCH₃, —C(O)(CH₂)₂NH₂; and —C(O)NH(CH₂)₂NHC(O)O-t-butyl;
R⁵ is H; and
R⁶ is is selected from the group consisting of H and bromo.

In embodiments, the present disclosure describes administering a compound of Formula (Ia):

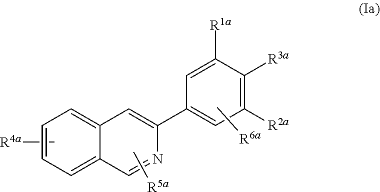

or a salt, solvate or hydrate thereof.

Each of substituents R^{1a} and R^{2a} of Formula (Ia) is independently selected from the group consisting of OH, OR^{7a}, and H, provided that at least one of R^{1a} and R^{2a} is OH or OR^{7a}.

Substituent R^{7a} of Formula (Ia) is selected from the group consisting of optionally substituted C₁₋₆ alkyl, optionally substituted C₃₋₆ cycloalkyl, optionally substituted aryl, optionally substituted aryl C₁₋₆ alkyl and acyl.

Substituent R^{3a} of Formula (Ia) is selected from the group consisting of optionally substituted C₁₋₆ alkyl, optionally substituted C₂₋₆ alkenyl, optionally substituted C₂₋₆ alkynyl, optionally substituted aryl, optionally substituted aryl C₁₋₆ alkyl, optionally substituted C₃₋₆ cycloalkyl, optionally substituted C₄₋₆ cycloalkenyl, halo, and optionally substituted heterocyclic ring.

Substituent R^{6a} of Formula (Ia) is selected from the group consisting of H, halo, hydroxyl, alkoxy, optionally substituted C₁₋₆ alkyl, halogenated alkyl, optionally substituted C₂₋₆ alkenyl, optionally substituted C₂₋₆ alkynyl, and optionally substituted aryl C₁₋₆ alkyl.

The substituent R^{5a} of Formula (Ia) is selected from the group consisting of H, halo, optionally substituted C₁₋₆ alkyl, —C(O)OR^{14a}, —C(O)NR^{15a}R^{16a}, optionally substituted aryl, and optionally substituted —C₁₋₆ alkylaryl.

Substituent R^{14a} of Formula (Ia) is independently selected from the group consisting of H, optionally substituted C₁₋₆ alkyl, optionally substituted C₃₋₆ cycloalkyl, optionally substituted aryl, and optionally substituted aryl C₁₋₆ alkyl.

Each of substituents R^{15a} and R^{16a} of Formula (Ia) is independently selected from the group consisting of H, optionally substituted C₁₋₆ alkyl, optionally substituted C₃₋₆ cycloalkyl, optionally substituted aryl, optionally substituted aryl C₁₋₆ alkyl, and optionally substituted C₃₋₆ cycloalkyl. Alternatively, R^{15a} and R^{16a} together with the nitrogen to which they are attached, forms a 5-7 membered cyclic saturated or unsaturated ring.

Subscript s′, of Formula (Ia) is an integer having a value of 0, 1 or 2.

Subscript t′ of Formula (Ia) is an integer having a value of 0 to 6.

Substituent R^{4a} of Formula (Ia) is selected from the group consisting of H, halo, cyano, optionally substituted C₁₋₆ alkyl, optionally substituted C₂₋₆ alkenyl, optionally substituted C₂₋₆ alkynyl, optionally substituted aryl, optionally substituted aryl C₁₋₆ alkyl, optionally substituted C₃₋₆ cycloalkyl, —(CR^{18a}R^{19a})ₜ COOR^{8a}, —(CR^{18a}R^{19a})ₜ OC(O)R^{8a}; —(CR^{18a}R^{19a})_{t′}NR⁹R¹⁰, —(CR^{18a}R^{19a})_{t′}C(O)NR^{9a}R^{10a}, —(CR^{18a}R^{19a})_{t′}NR^{9a}C(O)R^{8a}, —(CR^{18a}R^{19a})_{t′}S(O)₂NR^{9a}R^{10a}, —(CR^{18a}R^{19a})_{t′}COR^{11a}, —(CR^{18a}R^{19a})_{t′} CH(O), —(CR^{18a}R^{19a})_{t′}OR^{12a}, —(CR^{18a}R^{19a})_{t′}S(O)_{s′}R^{13a}; optionally substituted heterocyclic and optionally substituted heterocyclicC₁₋₆ alkyl.

Each of substituent R^{18a} and R^{19a} of Formula (Ia) is independently selected from the group consisting of H, optionally substituted C₁₋₆ alkyl, optionally substituted C₃₋₆ cycloalkyl, optionally substituted aryl, and optionally substituted aryl C₁₋₆ alkyl.

The substituent R^{8a} of Formula (Ia) is independently selected from the group consisting of H, optionally substituted C₁₋₆ alkyl, optionally substituted C₃₋₆ cycloalkyl, optionally substituted aryl, and optionally substituted aryl C₁₋₆ alkyl.

Each of substituents R^{9a} and R^{10a} of Formula (Ia) is independently selected from the group consisting of H, optionally substituted C₁₋₆ alkyl, optionally substituted C₃₋₆ cycloalkyl, optionally substituted aryl, and optionally substituted aryl C₁₋₆ alkyl. Alternatively, substituents R^{9a} and R^{10a} together with the nitrogen atom to which they are attached, form a 5-7 membered cyclic saturated or unsaturated ring.

Substituent R^{11a} of Formula (Ia) is independently selected from the group consisting of hydrogen, optionally substituted C₁₋₆ alkyl, optionally substituted C₂₋₆ alkenyl, optionally substituted C₂₋₆ alkynyl, optionally substituted aryl, optionally substituted aryl C₁₋₆ alkyl, optionally substituted C₃₋₆ cycloalkyl, —NR^{9a}R^{10a}, and —OR^{12a}.

Each of substituents R^{12a} and R^{13a} of Formula (Ia) is independently selected from the group consisting of H, optionally substituted alkyl, optionally substituted alkenyl, optionally substituted alkynyl, optionally substituted aryl, optionally substituted aryl C₁₋₆ alkyl, and optionally substituted C₃₋₆ cycloalkyl.

In some embodiments, the salt of Formula (Ia) is a pharmaceutically acceptable salt.

In some embodiments, each of R^{1a} and R^{2a} is independently OH. In some embodiments, one of R^{1a} and R^{2a} is OH and the other is H. In some embodiments, one of R^{1a} and R^{2a} is OH and the other is OR^{7a} and in some embodiments each of R^{1a} and R^{2a} is independently OR^{7a}, wherein each R^{7a} is independently selected from the group consisting of optionally substituted C₁₋₆ alkyl, optionally substituted C₃₋₆ cycloalkyl, optionally substituted aryl, optionally substituted aryl C₁₋₆ alkyl and acyl. In some embodiments, R^{7a} is an optionally substituted C₁₋₄ alkyl. In some embodiments, R^{7a} is methyl or ethyl.

In some embodiments, R^{3a} is selected from the group consisting of optionally substituted C₁₋₆ alkyl, and optionally substituted C₃₋₆ cycloalkyl. In some embodiments, R^{3a} is optionally substituted C₃₋₆ alkyl. In some embodiment, R^{3a} is isopropyl. In some embodiments, R^{3a} is optionally substituted C₃₋₆ cycloalkyl. In some embodiments, the cycloalkyl is cyclopentyl.

In some embodiments, R^{3a} is selected from the group consisting of optionally substituted C₃₋₆ alkyl, and optionally substituted C₃₋₆ cycloalkyl; and R^{1a} and R^{2a} are each independently OH. In some embodiments, R^{3a} is selected from the group consisting of optionally substituted C₃₋₆ alkyl, and optionally substituted C₃₋₆ cycloalkyl; and each of R^{1a} and R^{2a} is independently selected from the group consisting of OH, OR^{7a}, and H, provided that at least one of R^{1a} and R^{2a} is OH or OR^{7a}. In another embodiment, R^{3a} is selected from the group onsisting of optionally substituted C₃₋₆ alkyl and optionally substituted C₃₋₆ cycloalkyl; and one of R^{1a} and R^{2a} is OH and the other is H.

In some embodiments, each of R^{4a} and R^{5a} is independently selected from H and halo.

In some embodiments, R^{4a} is H. and R^{5a} is selected from the group consisting of H, halo, optionally substituted C₁₋₆ alkyl, C(O)OR^{14a}, and C(O)NR^{15a}R^{16a}. In some embodiments, R^{5a} is H, and R^{4a} is selected from the group consisting of H, halo, optionally substituted C₁₋₆ alkyl, C(O)OR^{14a}, and C(O)NR^{15a}R^{16a}.

In some embodiments, R^{3a} is selected from the group consisting of optionally substituted C₃₋₆ alkyl and optionally substituted C₃₋₆ cycloalkyl; both R^{1a} and R^{2a} are OH, and each of R^{4a} and R^{5a} is independently selected from H and halo.

In some embodiments, R^{3a} is an optionally substituted C₃₋₆ alkyl, each of R^{1a} and R^{2a} is OH, and each of R^{4a} and R^{5a} is independently H or halo.

In some embodiments, R^{6a} is independently selected from the group consisting of H, halo, hydroxyl, C₁₋₆ alkoxy, optionally substituted C₁₋₆ alkyl, halogenated alkyl, optionally substituted C₂₋₆ alkenyl, optionally substituted C₂₋₆ alkynyl, and optionally substituted aryl C₁₋₆ alkyl. In some embodiments, R^{6a} is selected from the group consisting of H, halo, hydroxyl, C₁₋₃ alkoxy, optionally substituted C₁₋₃ alkyl, and halogenated alkyl. In some embodiments, R^{6a} is selected from the group consisting of H and halo. In some embodiments, R^{6a} is H.

In some embodiments, R^{6a} is selected from the group consisting of H and halo; R^{3a} is an optionally substituted C₃₋₆ alkyl, each of R^{1a} and R^{2a} is OH, and each of R^{4a} and R^{5a} is independently selected from the group consisting of H and halo.

In some embodiments, t is 0. In some embodiments, t is 0, 1, 2, or 3.

Some embodiments of the invention are directed to a compound of selected from the group consisting of.

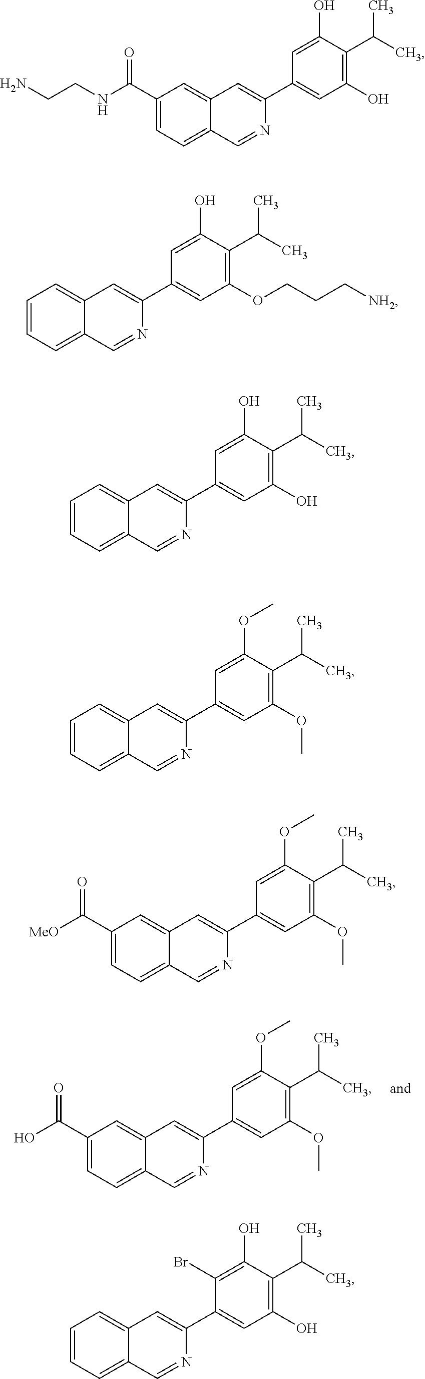

or a salt, solvate or hydrate thereof.

Some embodiments of the invention are directed to 2-isopropyl-5-(isoquinolin-3-yl)benzene-1,3-diol:

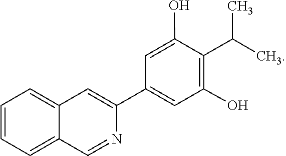 [SMILES: CC(C)c1c(O)cc(-c2cc3ccccc3cn2)cc1O]

or a pharmaceutically acceptable salt, solvate or hydrate thereof.

In some embodiments the 2-isopropyl-5-(isoquinolin-3-yl)benzene-1,3-diol, is a solid. In some embodiments the solid is a crystalline solid. In some embodiments the solid is an amorphous solid. In some embodiments the 2-isopropyl-5-(isoquinolin-3-yl)benzene-1,3-diol is a non-solvated crystal. Some embodiments describe isolated 2-isopropyl-5-(isoquinolin-3-yl)benzene-1,3-diol. In some embodiments the 2-isopropyl-5-(isoquinolin-3-yl)benzene-1,3-diol is a hydrate. In some embodiments the 2-isopropyl-5-(isoquinolin-3-yl)benzene-1,3-diol is an organic solvate. Some embodiments describe isolated 2-isopropyl-5-(isoquinolin-3-yl)benzene-1,3-diol organic solvate. Some embodiments describe 2-isopropyl-5-(isoquinolin-3-yl)benzene-1,3-diol acetonitrile/water solvate. Some embodiments describe 2-isopropyl-5-(isoquinolin-3-yl)benzene-1,3-diol acetone solvate. Some embodiments describe 2-isopropyl-5-(isoquinolin-3-yl)benzene-1,3-diol N,N-dimethylformamide solvate. Some embodiments describe 2-isopropyl-5-(isoquinolin-3-yl)benzene-1,3-diol 1,4-dioxane/water solvate. Some embodiments describe 2-isopropyl-5-(isoquinolin-3-yl)benzene-1,3-diol butanone solvate. Some embodiments describe 2-isopropyl-5-(isoquinolin-3-yl)benzene-1,3-diol tetrahydrofuran/water solvate. Some embodiments describe 2-isopropyl-5-(isoquinolin-3-yl)benzene-1,3-diol ethylacetate solvate. Some embodiments describe 2-isopropyl-5-(isoquinolin-3-yl)benzene-1,3-diol dimethylcarbonate solvate. Some embodiments describe 2-isopropyl-5-(isoquinolin-3-yl)benzene-1,3-diol dimethylsulfoxide solvate. Some embodiments describe 2-isopropyl-5-(isoquinolin-3-yl)benzene-1,3-diol 1-butanol solvate. Some embodiments describe 2-isopropyl-5-(isoquinolin-3-yl)benzene-1,3-diol tetrahydrofuran solvate. Some embodiments describe 2-isopropyl-5-(isoquinolin-3-yl)benzene-1,3-diol methyl t-butyl ether solvate.

The invention also includes various isomers of a compound according to any embodiment described herein, and mixtures thereof. Isomers are compounds that have the same composition and molecular weight but differ in physical and/or chemical properties. The structural difference may be in constitution (geometric isomers) or in the ability to rotate the plane of polarized light (stereoisomers). A compound according to any embodiment described herein, may contain one or more asymmetric centers, also referred to as chiral centers, and may, therefore, exist as individual enantiomers, diastereomers, or other stereoisomeric forms, or as mixtures thereof. All such isomeric forms are included within the present invention, including mixtures thereof. Chiral centers may also be present in a substituent such as an alkyl group. Where the stereochemistry of a chiral center present in a formula, or in any chemical structure illustrated herein, is not specified the structure is intended to encompass any stereoisomer and all mixtures thereof. Thus, a compound according to any embodiment described herein, containing one or more chiral centers may be used as racemic mixtures, enantiomerically enriched mixtures, or as enantiomerically pure individual stereoisomers. A mixture containing unequal portions of the enantiomers is described as having an “enantiomeric excess” (ee) of either the R or S compound. The excess of one enantiomer in a mixture is often described with a % enantiomeric excess. The ratio of enantiomers can also be defined by “optical purity” wherein the degree at which the mixture of enantiomers rotates plane polarized light is compared to the individual optically pure R and S compounds. The compounds can also be a substantially pure (+) or (−) enantiomer of the compounds described herein. In some embodiments, a composition can include a substantially pure enantiomer of a compound according to any embodiment described herein, that is at least 90%, 91%, 92%, 93%, 94%, 95%, 96%, 97%, 98%, or 99% of one enantiomer. In certain embodiments, a composition may include a substantially pure enantiomer of a compound according to any embodiment described herein, that is at least 99.5% of one enantiomer.

Individual stereoisomers of a compound according to any embodiment described herein, which contain one or more asymmetric centers may be resolved by methods known to those skilled in the art. For example, such resolution may be carried out (1) by formation of diastereoisomeric salts, complexes or other derivatives; (2) by selective reaction with a stereoisomer specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation. Further embodiments include prodrugs of a compound according to any embodiment described herein, i.e., compounds which release an active compound according to any of the embodiments described herein, in vivo when administered to a mammalian subject. A prodrug is a pharmacologically active or more typically an inactive compound that is converted into a pharmacologically active agent by a metabolic transformation. Prodrugs of a compound according to any embodiment described herein, are prepared by modifying functional groups present in the compound in such a way that the modifications may be cleaved in vivo to release the parent compound. In vivo, a prodrug readily undergoes chemical changes under physiological conditions (e.g., are hydrolyzed or acted on by naturally occurring enzyme(s)) resulting in liberation of the pharmacologically active agent. Prodrugs include compounds according to any embodiment described herein, wherein a hydroxyl, amino, or carboxy group is bonded to any group that may be cleaved in vivo to regenerate the free hydroxyl, amino or carboxy group, respectively. Examples of prodrugs include, but are not limited to esters (e.g., acetate, formate, and benzoate derivatives) of compounds according to any embodiment described herein, or any other derivative which upon being brought to the physiological pH or through enzyme action is converted to the active parent drug. Conventional procedures for the selection and preparation of suitable prodrug derivatives are described in the art.

Certain of the compounds of the invention may form salts with one or more equivalents of an acid (if the compound contains a basic moiety) or a base (if the compound contains an acidic moiety). The present invention includes within its scope all possible stoichiometric and non-stoichiometric salt forms.

When a compound of the invention contains a basic moiety, a desired salt form may be prepared by any suitable method known in the art, including treatment of the free base with an inorganic acid, such as hydrochloric acid, hydrobromic acid, sulfuric acid, nitric acid, phosphoric acid, and the like; or with an organic acid, such as acetic acid, trifluoroacetic acid, maleic acid, succinic acid, mandelic acid, fumaric acid, malonic acid, pyruvic acid, oxalic acid, glycolic acid, salicylic acid, and the like; or with a pyranosidyl acid, such as glucuronic acid or galacturonic acid; or with an alpha-hydroxy acid, such as citric acid or tartaric acid; or with an amino acid, such as aspartic acid or glutamic acid; or with an aromatic acid, such as benzoic acid or cinnamic acid; or with a sulfonic acid, such as p-toluenesulfonic acid, methanesulfonic acid, ethanesulfonic acid or the like.

Suitable addition salts are formed from acids which form non-toxic salts and examples include acetate, p-aminobenzoate, ascorbate, aspartate, benzenesulfonate, benzoate, bicarbonate, bismethylenesalicylate, bisulfate, bitartrate, borate, calcium edetate, camsylate, carbonate, clavulanate, citrate, cyclohexylsulfamate, edetate, edisylate, estolate, esylate, ethanedisulfonate, ethanesulfonate, formate, fumarate, gluceptate, gluconate, glutamate, glycollate, glycollylarsanilate, hexylresorcinate, hydrobromide, hydrochloride, dihydrochloride, hydrofumarate, hydrogen phosphate, hydroiodide, hydromaleate, hydrosuccinate, hydroxynaphthoate, isethionate, itaconate, lactate, lactobionate, laurate, malate, maleate, mandelate, mesylate, methylsulfate, monopotassium maleate, mucate, napsylate, nitrate, N-methylglucamine, oxalate, oxaloacetate, pamoate (embonate), palmate, palmitate, pantothenate, phosphate/diphosphate, pyruvate, polygalacturonate, propionate, saccharate, salicylate, stearate, subacetate, succinate, sulfate, tannate, tartrate, teoclate, tosylate, triethiodide, trifluoroacetate and valerate.

Other exemplary acid addition salts include pyrosulfate, sulfite, bisulfite, decanoate, caprylate, acrylate, isobutyrate, caproate, heptanoate, propiolate, oxalate, malonate, suberate, sebacate, butyne-1,4-dioate, hexyne-1,6-dioate, chlorobenzoate, methylbenzoate, dinitrobenzoate, hydroxybenzoate, methoxybenzoate, phthalate, phenylacetate, phenylpropionate, phenylbutrate, lactate, γ-hydroxybutyrate, mandelate, and sulfonates, such as xylenesulfonate, propanesulfonate, naphthalene-1-sulfonate and naphthalene-2-sulfonate.

If an inventive basic compound is isolated as a salt, the corresponding free base form of that compound may be prepared by any suitable method known to the art, including treatment of the salt with an inorganic or organic base, suitably an inorganic or organic base having a higher pKₐ than the free base form of the compound.

When a compound of the invention contains an acidic moiety, a desired salt may be prepared by any suitable method known to the art, including treatment of the free acid with an inorganic or organic base, such as an amine (primary, secondary, or tertiary), an alkali metal or alkaline earth metal hydroxide, or the like. Illustrative examples of suitable salts include celitec salts derived from amino acids such as glycine and arginine, ammonia; primary, secondary, and tertiary amines, and cyclic amines, such as N-methyl-D-glucamine, diethylamine, isopropylamine, trimethylamine, ethylene diamine, dicyclohexylamine, ethanolamine, piperidine, morpholine, and piperazine; as well as inorganic salts derived from sodium, calcium, potassium, magnesium, manganese, iron, copper, zinc, aluminum, and lithium.

Those compounds of the invention having both a basic and acidic moiety may be in the form of zwitterions, acid-addition salt of the basic moiety or base salts of the acidic moiety.

Because of their potential use in medicine, the salts of the compounds of the invention are preferably pharmaceutically acceptable salts. Suitable pharmaceutically acceptable salts are known to those of skill in the art.

These pharmaceutically acceptable salts may be prepared in situ during the final isolation and purification of the compound, or by separately treating the purified compound in its free acid or free base form with a suitable base or acid, respectively.

In certain embodiments, compounds of the invention may contain an acidic functional group and are, therefore, capable of forming pharmaceutically acceptable base addition salts by treatment with a suitable base. Examples of such bases include, but are not limited to, a) hydroxides, carbonates, and bicarbonates of sodium, potassium, lithium, calcium, magnesium, aluminum, and zinc; and b) primary, secondary, and tertiary amines including aliphatic amines, aromatic amines, aliphatic diamines, and hydroxy alkylamines such as methylamine, ethylamine, 2-hydroxyethylamine, diethylamine, triethylamine, ethylenediamine, ethanolamine, diethanolamine, and cyclohexylamine.

In certain embodiments, compounds of the invention may contain a basic functional group and are therefore capable of forming pharmaceutically acceptable acid addition salts by treatment with a suitable acid. Suitable acids include pharmaceutically acceptable inorganic acids and organic acids. Representative pharmaceutically acceptable acids include hydrogen chloride, hydrogen bromide, nitric acid, sulfuric acid, sulfonic acid, phosphoric acid, acetic acid, hydroxyacetic acid, phenylacetic acid, propionic acid, butyric acid, valeric acid, maleic acid, acrylic acid, fumaric acid, succinic acid, malic acid, malonic acid, tartaric acid, citric acid, salicylic acid, benzoic acid, tannic acid, formic acid, stearic acid, lactic acid, ascorbic acid, methylsulfonic acid, p-toluenesulfonic acid, oleic acid, lauric acid, and the like.

This invention also provides for the conversion of one pharmaceutically acceptable salt of a compound of the invention into another pharmaceutically acceptable salt of a compound of this invention.

A compound according to any embodiment described herein, may exist in solid or liquid form. In the solid state, it may exist in crystalline or non-crystalline form, or as a mixture thereof. The skilled artisan will appreciate that pharmaceutically acceptable solvates may be formed from crystalline compounds wherein solvent molecules are incorporated into the crystalline lattice during crystallization. Solvates may involve non-aqueous solvents such as, but not limited to, ethanol, isopropanol, DMSO, acetic acid, ethanolamine, or ethyl acetate, or they may involve water as the solvent that is incorporated into the crystalline lattice. Solvates wherein water is the solvent incorporated into the crystalline lattice are typically referred to as “hydrates.” Hydrates include stoichiometric hydrates as well as compositions containing variable amounts of water. The invention includes all such solvates.

For solvates of a compound according to any embodiment described herein, including solvates of salts of a compound according to any embodiment described herein, that are in crystalline form, the skilled artisan will appreciate that pharmaceutically acceptable solvates may be formed wherein solvent molecules are incorporated into the crystalline lattice during crystallization. Solvates may involve non-aqueous solvents such as ethanol, isopropanol, DMSO, acetic acid, ethanolamine, and EtOAc, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

The skilled artisan will further appreciate that a compound according to any embodiment described herein that exists in crystalline form, including the various solvates thereof, may exhibit polymorphism (i.e., the capacity to occur in different crystalline structures). These different crystalline forms are typically known as “polymorphs.” The invention includes all such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

The subject invention also includes isotopically-labelled compounds, which are identical to those recited in compounds of the invention and following, but for the fact that one or more atoms are replaced by an atom having an atomic mass or mass number different from the atomic mass or mass number usually found in nature. Examples of isotopes that can be incorporated into compounds of the invention and pharmaceutically acceptable salts, solvates or hydrates thereof, include isotopes of hydrogen, carbon, nitrogen, oxygen, phosphorous, sulphur, fluorine, iodine, and chlorine, such as ²H, ³H, ¹¹C, ¹³C, ¹⁴C, ¹⁵N, ¹⁷O, ¹⁸O, ³¹P, ³²P, ³⁵S, ¹⁸F, ³⁶Cl, ¹²³I and ¹²⁵I.

Compounds according to any embodiment described herein and pharmaceutically acceptable salts, solvates or hydrates of said compounds that contain the aforementioned isotopes and/or other isotopes of other atoms are within the scope of the present invention. Isotopically-labelled compounds of the present invention, for example those into which radioactive isotopes such as ³H, ⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ¹⁸F isotopes are particularly useful in PET (positron emission tomography), and ¹²⁵I isotopes are particularly useful in SPECT (single photon emission computerized tomography), and are also useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds according to any embodiment described herein, can generally be prepared by carrying out the procedures disclosed in the Schemes and/or in the Examples below, by substituting a readily available isotopically labeled reagent for a non-isotopically labeled reagent.

The invention also embraces isolated compounds. An isolated compound refers to a compound which represents at least 10%, preferably at least 20%, more preferably at least 50% and most preferably at least 80% of the compound present in the mixture.

Because the compounds according to any embodiment described herein are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). In some embodiments, a compound according to any embodiment described herein is at least 90%, at least 91%, at least 92%, at least 93%, at least 94%, at least 95%, at least 96%, at least 97%, at least 98%, or at least 99% pure. Impure preparations of the compounds may be used for preparing the more pure forms used in the pharmaceutical compositions.

### Pharmaceutical Compositions Administered in Method Embodiments Described Herein

Some embodiments describe administering a pharmaceutical composition comprising: a compound according to any embodiment described herein, a pharmaceutically acceptable salt thereof, a solvate thereof, or a hydrate thereof; and a pharmaceutically acceptable carrier or diluent. The pharmaceutical compositions can be prepared in a manner well known in the pharmaceutical arts, and can be administered by a variety of routes, depending upon whether local or systemic treatment is desired and upon the area to be treated.

While it is possible that a compound as described in any embodiment herein, may be administered as the bulk substance, it is preferable to present the compound in a pharmaceutical formulation, e.g., wherein the active agent is in an admixture with a pharmaceutically acceptable carrier selected with regard to the intended route of administration and standard pharmaceutical practice.

In particular, the disclosure provides administering a pharmaceutical composition comprising a therapeutically effective amount of at least one compound according to any embodiment described herein, and optionally, a pharmaceutically acceptable carrier.

Some embodiments describe administering a pharmaceutical composition comprising 2-Isopropyl-5-(isoquinolin-3-yl)benzene-1,3-diol:

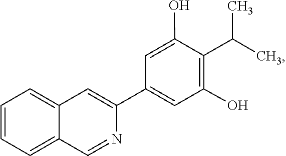 [SMILES: CC(C)c1c(O)cc(-c2cc3ccccc3cn2)cc1O]

or a pharmaceutically acceptable salt, solvate or hydrate thereof and a pharmaceutically acceptable carrier or diluent.

Some embodiments describe administering a pharmaceutical composition comprising N-(2-aminoethyl)-3-[3,5-dihydroxy-4-(propan-2-yl)phenyl]isoquinoline-6-carboxamide:

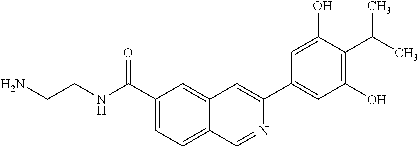 [SMILES: CC(C)c1c(O)cc(-c2cc3cc(C(=O)NCCN)ccc3cn2)cc1O]

or a pharmaceutically acceptable salt, solvate or hydrate thereof and a pharmaceutically acceptable carrier or diluent.

Some embodiments describe administering a pharmaceutical composition comprising 3-(3-aminopropoxy)-5-(isoquinolin-3-yl)-2-(propan-2-yl)phenol:

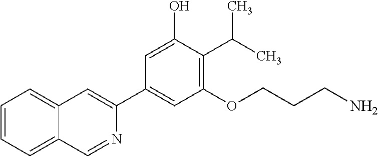 [SMILES: CC(C)c1c(O)cc(-c2cc3ccccc3cn2)cc1OCCCN]

or a pharmaceutically acceptable salt, solvate or hydrate thereof and a pharmaceutically acceptable carrier or diluent. In one embodiment, the salt is trifluroacetic acid salt:

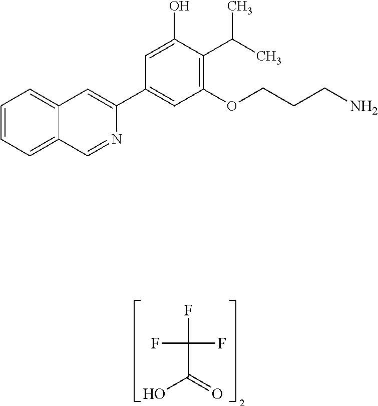

### Methods of Treatment

AhR activation has been shown to enhance regulatory B-cell activity to suppress inflammationl, thus, the compounds described herein are proposed to inhibit B-cell driven pathology including those due to deficient regulatory B-cell activity or pathology driven B-cell differentiation including autoantibody and/or pathogenic antibody production, including, but not limited to examples of which include Pemphigus foliaceus, Pemphigus vulgaris, paraneoplastic pemphigus, bullous pemphigoid, epidermolysis bullosa acquisita, lupus erythematosus, dermatomyositis, myasthenia gravis, goodpadture's syndrome, autoimmune thyroiditis.

AhR agonism inhibits activation and migration of polymorphonuclear leukocytes thus, the compounds described herein are proposed to inhibit neutrophilic disorders or diseases in which polymorphonuclear leukocytes contribute to the pathology, including, but not limited to examples of which include Sweet's syndrome, pustular psoriasis, pyoderma gangrenosum, hidradenitis suppuritiva, leukocytoclastic vasculitis Wegener's Granulomatosis, and neutrophilic dermatoses.

Ahr agonists have been demonstrated to promote long term graft acceptance in vivo through a mechanism dependent on AHR signaling in dendritic cells/antigen presenting cells and T cells and in vitro, the AhR agonists inhibit the activation of monocyte-derived DCs (moDCs)/antigen presenting cells, including their production of inflammatory cytokines like IL-6 and upregulation of costimulatory molecules. Thus, the compounds described herein are proposed to inhibit inflammation and inflammatory disorders in which antigen presenting cells stimulate immune responses. Such diseases include, but are not limited to, allergic and autoimmune diseases including inflammatory airway disease, allergic rhinitis, autoimmune blistering diseases including Pemphigus vulgaris and Pemphigus foliaceus, and bullous pemphigoid, Schamberg's disease or other pigmented purpuric dermatoses (PPDs), inflammatory joint disease including lupus vulgaris, and dermatomyositis.

Mast cells express AhR and their activity and histamine release is inhibited by repetitive stimulation of AhR6, thus the compounds described herein are proposed to inhibit disorders of mast cell activation including, but not limited to, examples of which include mastocytosis, urticaria, rosacea, fibromyalgia, chronic pain syndromes, chronic pruritis, chronic itch, and allergic rhinitis.

AhR has a normal physiological role is vascular growth and function11. Studies have demonstrated suppression of endothelial cell proliferation and stimulation of cancer cell death leading to the suppression of tumor growth. The compounds described herein are proposed to suppress angiogenesis which may lead to tumor growth inhibition. Thus, the compounds described herein are proposed to inhibit angiogenesis and related disorders of the stromal environment, vascular function and arteriosclerosis and tumor growth which may include both benign and malignant.

AhR is a key regulator in innate lymphoid cells and has been implicated in the development and/or function of ILCs—specifically type 3 (iLC3) which are predominantly found in the GI tract and relatively rate in other tissues. ILC3s respond most notably IL-23, IL-1β, IL-15, and IL-18. These cells are known to have the following effector molecules (IL-22, IFNγ, GM-CSF, and IL-17). ILC3 are involved in the clearance of bacterial and fungal infection, control of enteric virus infection, and maintenance of the microbiota homeostasis—ILC3 have been shown to participate in ILC-driven colitis whereby perturbation of the iLC3 composition correlates with disease severity. The compounds described herein are proposed to inhibit disorders associated with ILC3 dysregulation in the gut.

ILC2 cells have been implicated in asthma since ILC2 cells produce large amounts of IL-5 and IL-13. Studies have shown that ILC2 increase in the airways of severe asthma patients. Although the precise function of ILC2 remains to be determined, several AhR ligands have been shown to suppress allergic airway inflammation in mouse models via suppression of type 2 cytokines (IL-4 and IL-5), eosinophilia, and specific IgE expression. ILC2 populations within the lung also have the ability to produce IL-17 and this specific subset may play a role in the pathology of asthma.

AhR has been demonstrated to promote the antitumor activity of NK cells and while the exact function of AhR in ILC-mediated tumor immunology remains largely unknown, AhR agonism may promote and thus, the compounds described herein are proposed to result in antitumor action in ILC and NK cells.

AhR has been demonstrated to modulate the function of macrophages and plays a role in the phagocytosis of apoptotic cells and is a key regulator of macrophage responsiveness and homeostasis during inflammatory responses. Such diseases where AhR modulation of macrophages may contribute, and thus, the compounds described herein are proposed to treat, include, but are not limited to, allergic and autoimmune diseases including inflammatory airway disease, allergic rhinitis, Schamberg's disease or other pigmented purpuric dermatoses (PPDs), and inflammatory joint disease.

Furthermore, the interaction amongst these various immune cells contributes to the inflammatory cascade that drives inflammatory and autoimmune disease. Thus, AhR agonism acts both at individual cell types and collectively via the interactions amongst these cells that drive inflammation. Thus, the compounds described herein are proposed to additionally inhibit inflammatory and autoimmune pathology by acting on the above cited cell specific targets as well as through inflammatory networks comprised of these cells.

The present invention thus provides methods for the treatment of disorders associated with the abovementioned diseases or disorders, comprising the step of administering to a subject in need thereof at least one compound, as described in any embodiment herein, in an amount effective therefore.

In some embodiments, the disclosure provides methods of preventing or treating an AhR mediated disease, as noted above, comprising administering to said subject an effective amount of a compound according to any embodiment described herein, or pharmaceutically acceptable salt, solvate or hydrate thereof; or a pharmaceutical composition according to any embodiment described herein. In some embodiments the compound is a compound of Formula (I) or Formula (Ia) or a pharmaceutically acceptable salt, solvate or hydrate thereof. In some embodiments the compound is a compound of Formula (I) or Formula (Ia), or a pharmaceutically acceptable salt, solvate or hydrate thereof in combination with another therapeutic agent. In some embodiments the compound is 2-isopropyl-5-(isoquinolin-3-yl)benzene-1,3-diol, or a pharmaceutically acceptable salt, solvate or hydrate thereof. In some embodiments the compound is 2-isopropyl-5-(isoquinolin-3-yl)benzene-1,3-diol, or a pharmaceutically acceptable salt, solvate or hydrate thereof in combination with another therapeutic agent.

Some embodiments describe a method for treating or preventing disorders such as: Pemphigus foliaceus, Pemphigus vulgaris, paraneoplastic pemphigus, bullous pemphigoid, epidermolysis bullosa acquisita, lupus erythematosus, dermatomyositis, myasthenia gravis, goodpadture's syndrome, and autoimmune thyroiditis; Sweet's syndrome, pustular psoriasis, pyoderma gangrenosum, hidradenitis suppuritiva, leukocytoclastic vasculitis Wegener's Granulomatosis and neutrophilic dermatoses; allergic diseases, autoimmune diseases, inflammatory airway disease, allergic rhinitis, autoimmune blistering diseases, Pemphigus vulgaris and Pemphigus foliaceus, bullous pemphigoid, inflammatory joint disease, Schamberg's disease or other pigmented purpuric dermatoses (PPDs), lupus vulgaris and dermatomyositis; mastocytosis, urticaria, rosacea, fibromyalgia, chronic pain syndromes, chronic pruritis, chronic itch, allergic rhinitis; arteriosclerosis, benign tumor growth and malignant tumor growth, inflammatory airway disease, allergic rhinitis, Schamberg's disease or other pigmented purpuric dermatoses (PPDs), inflammatory joint disease; comprising administering an effective amount of a compound according to any embodiment described herein, or pharmaceutically acceptable salt thereof, or a pharmaceutical composition, according to any embodiment described herein.

Some embodiments describe a method for treating or preventing an AhR mediated disease selected from Pemphigus foliaceus, Pemphigus vulgaris, paraneoplastic pemphigus, bullous pemphigoid, epidermolysis bullosa acquisita, lupus erythematosus, dermatomyositis, myasthenia gravis, goodpadture's syndrome, and autoimmune thyroiditis in a subject in need thereof, comprising administering to said subject an effective amount of a compound or pharmaceutical composition according to any embodiment described herein, or pharmaceutically acceptable salt, solvate or hydrate thereof. In some embodiments the compound is a compound of Formula (I) or Formula (Ia) or a pharmaceutically acceptable salt, solvate or hydrate thereof. In some embodiments the compound is a compound of Formula (I) or Formula (Ia) or a pharmaceutically acceptable salt, solvate or hydrate thereof in combination with another therapeutic agent. In some embodiments the compound is 2-isopropyl-5-(isoquinolin-3-yl)benzene-1,3-diol, or a pharmaceutically acceptable salt, solvate or hydrate thereof. In some embodiments the compound is 2-isopropyl-5-(isoquinolin-3-yl)benzene-1,3-diol, or a pharmaceutically acceptable salt, solvate or hydrate thereof in combination with another therapeutic agent.

Some embodiments describe a method for treating or preventing an AhR mediated disease selected from Sweet's syndrome, pustular psoriasis, pyoderma gangrenosum, hidradenitis suppuritiva, leukocytoclastic vasculitis Wegener's Granulomatosis and neutrophilic dermatoses in a subject in need thereof, comprising administering to said subject an effective amount of a compound or pharmaceutical composition according to any embodiment described herein, or pharmaceutically acceptable salt, solvate or hydrate thereof. In some embodiments the compound is a compound of Formula (I) or Formula (Ia) or a pharmaceutically acceptable salt, solvate or hydrate thereof. In some embodiments the compound is a compound of Formula (I) or Formula (Ia) or pharmaceutically acceptable salt, solvate or hydrate thereof in combination with another therapeutic agent. In some embodiments the compound is 2-isopropyl-5-(isoquinolin-3-yl)benzene-1,3-diol, or a pharmaceutically acceptable salt, solvate or hydrate thereof. In some embodiments the compound is 2-isopropyl-5-(isoquinolin-3-yl)benzene-1,3-diol, or a pharmaceutically acceptable salt, solvate or hydrate thereof in combination with another therapeutic agent.

Some embodiments describe a method for treating or preventing an AhR mediated disease selected from allergic diseases, autoimmune diseases, inflammatory airway disease, allergic rhinitis, autoimmune blistering diseases, Pemphigus vulgaris and Pemphigus foliaceus, bullous pemphigoid, Schamberg's disease or other pigmented purpuric dermatoses (PPDs), inflammatory joint disease, lupus vulgaris and dermatomyositis in a subject in need thereof, comprising administering to said subject an effective amount of a compound or pharmaceutical composition according to any embodiment described herein, or pharmaceutically acceptable salt, solvate or hydrate thereof. In some embodiments the compound is a compound of Formula (I) or Formula (Ia) or a pharmaceutically acceptable salt, solvate or hydrate thereof. In some embodiments the compound is a compound of Formula (I) or Formula (Ia) or pharmaceutically acceptable salt, solvate or hydrate thereof in combination with another therapeutic agent. In some embodiments the compound is 2-isopropyl-5-(isoquinolin-3-yl)benzene-1,3-diol, or a pharmaceutically acceptable salt, solvate or hydrate thereof. In some embodiments the compound is 2-isopropyl-5-(isoquinolin-3-yl)benzene-1,3-diol, or a pharmaceutically acceptable salt, solvate or hydrate thereof in combination with another therapeutic agent.

Some embodiments describe a method for treating or preventing an AhR mediated disease selected from mastocytosis, urticaria, rosacea, fibromyalgia, chronic pain syndromes, chronic pruritis, chronic itch, and allergic rhinitis, in a subject in need thereof, comprising administering to said subject an effective amount of a compound or pharmaceutical composition according to any embodiment described herein, or pharmaceutically acceptable salt, solvate or hydrate thereof. In some embodiments the compound is a compound of Formula (I) or Formula (Ia) or a pharmaceutically acceptable salt, solvate or hydrate thereof. In some embodiments the compound is a compound of Formula (I) or Formula (Ia) or pharmaceutically acceptable salt, solvate or hydrate thereof in combination with another therapeutic agent. In some embodiments the compound is 2-isopropyl-5-(isoquinolin-3-yl)benzene-1,3-diol, or a pharmaceutically acceptable salt, solvate or hydrate thereof. In some embodiments the compound is 2-isopropyl-5-(isoquinolin-3-yl)benzene-1,3-diol, or a pharmaceutically acceptable salt, solvate or hydrate thereof in combination with another therapeutic agent.

Some embodiments describe a method for treating or preventing an AhR mediated disease selected from inflammatory airway disease, Schamberg's disease, pigmented purpuric dermatoses (PPDs), and inflammatory joint disease in a subject in need thereof, comprising administering to said subject an effective amount of a compound or pharmaceutical composition according to any embodiment described herein, or pharmaceutically acceptable salt, solvate or hydrate thereof. In some embodiments the compound is a compound of Formula (I) or Formula (Ia) or a pharmaceutically acceptable salt, solvate or hydrate thereof. In some embodiments the compound is a compound of Formula (I) or Formula (Ia) or pharmaceutically acceptable salt, solvate or hydrate thereof in combination with another therapeutic agent. In some embodiments the compound is 2-isopropyl-5-(isoquinolin-3-yl)benzene-1,3-diol, or a pharmaceutically acceptable salt, solvate or hydrate thereof. In some embodiments the compound is 2-isopropyl-5-(isoquinolin-3-yl)benzene-1,3-diol, or a pharmaceutically acceptable salt, solvate or hydrate thereof in combination with another therapeutic agent.

Some embodiments describe a method for treating or preventing an AhR mediated disease in a subject in need thereof, comprising administering to said subject an effective amount of a compound or pharmaceutical composition according to any embodiment described herein, or pharmaceutically acceptable salt, solvate or hydrate thereof. In some embodiments the compound is a compound of Formula (I) or Formula (Ia) or a pharmaceutically acceptable salt, solvate or hydrate thereof. In some embodiments the compound is a compound of Formula (I) or Formula (Ia) or pharmaceutically acceptable salt, solvate or hydrate thereof in combination with another therapeutic agent. In some embodiments the compound is 2-isopropyl-5-(isoquinolin-3-yl)benzene-1,3-diol, or a pharmaceutically acceptable salt, solvate or hydrate thereof. In some embodiments the compound is 2-isopropyl-5-(isoquinolin-3-yl)benzene-1,3-diol, or a pharmaceutically acceptable salt, solvate or hydrate thereof in combination with another therapeutic agent.

Some embodiments describe a method for treating or preventing an AhR mediated disease selected from inflammatory airway disease, allergic rhinitis, Schamberg's disease or other pigmented purpuric dermatoses (PPDs), and inflammatory joint disease in a subject in need thereof, comprising administering to said subject an effective amount of a compound or pharmaceutical composition according to any embodiment described herein, or pharmaceutically acceptable salt, solvate or hydrate thereof. In some embodiments the compound is a compound of Formula (I) or Formula (Ia) or a pharmaceutically acceptable salt, solvate or hydrate thereof. In some embodiments the compound is a compound of Formula (I) or Formula (Ia) or pharmaceutically acceptable salt, solvate or hydrate thereof in combination with another therapeutic agent. In some embodiments the compound is 2-isopropyl-5-(isoquinolin-3-yl)benzene-1,3-diol, or a pharmaceutically acceptable salt, solvate or hydrate thereof. In some embodiments the compound is 2-isopropyl-5-(isoquinolin-3-yl)benzene-1,3-diol, or a pharmaceutically acceptable salt, solvate or hydrate thereof in combination with another therapeutic agent.

Some embodiments describe a method for treating or preventing an AhR mediated disease selected from skin inflammation, contact dermatitis due to poison ivy, eczema, chronic atopic dermatitis, acute flares of atopic dermatitis, psoriasis vulgaris plaques, mild-to-moderate psoriasis, pustulosis palmoplanteris, erythematous papules and plaques, vesiculation, exudation and crusting, erosion and scaling, acute radiation dermatitis, chronic radiation dermatitis, acute erythema, sale, desquamation, fibrosis, telangiectasias, morphea, skin atrophy, inflammatory mucosal conditions, inflammatory mucosal conditions caused by cancer radiation or chemotherapy treatment, oral mucositis, oral mucositis induced by head and/or neck cancer radiation or chemotherapy treatment, itching in atopic dermatitis, intense and/or constant itching, skin barrier dysfunction, deficient skin barrier function, visible skin lesions, acute flares, T-cell mediated hypersensitivity diseases, contact hypersensitivity, delayed-type hypersensivity, autoimmune alopecia, alopecia areata, uticaria, scleroderma, mycosis fungoides, chronic actinic dermatitis, epidermis thickening induced by imiquimod, and inflammatory skin lesions, in a subject in need thereof, comprising administering to said subject an effective amount of a compound or pharmaceutical composition according to any embodiment described herein, or pharmaceutically acceptable salt, solvate or hydrate thereof. In some embodiments the compound is a compound of Formula (I) or Formula (Ia) or a pharmaceutically acceptable salt, solvate or hydrate thereof. In some embodiments the compound is a compound of Formula (I) or Formula (Ia) or pharmaceutically acceptable salt, solvate or hydrate thereof in combination with another therapeutic agent. In some embodiments the compound is 2-isopropyl-5-(isoquinolin-3-yl)benzene-1,3-diol, or a pharmaceutically acceptable salt, solvate or hydrate thereof. In some embodiments the compound is 2-isopropyl-5-(isoquinolin-3-yl)benzene-1,3-diol, or a pharmaceutically acceptable salt, solvate or hydrate thereof in combination with another therapeutic agent.

Some embodiments describe a method for treating or preventing an AhR mediated disease selected from rejection of a clinical transplant (such as an organ transplant, acute transplant, heterograft, or homograft; ischemic or reperfusion injury; ischemic or reperfusion injury incurred during organ transplantation, myocardial infarction, stroke or other causes; in a subject in need thereof, comprising administering to said subject an effective amount of a compound or pharmaceutical composition according to any embodiment described herein, or pharmaceutically acceptable salt, solvate or hydrate thereof. In some embodiments the compound is a compound of Formula (I) or Formula (Ia) or a pharmaceutically acceptable salt, solvate or hydrate thereof. In some embodiments the compound is a compound of Formula (I) or Formula (Ia) or pharmaceutically acceptable salt, solvate or hydrate thereof in combination with another therapeutic agent. In some embodiments the compound is 2-isopropyl-5-(isoquinolin-3-yl)benzene-1,3-diol, or a pharmaceutically acceptable salt, solvate or hydrate thereof. In some embodiments the compound is 2-isopropyl-5-(isoquinolin-3-yl)benzene-1,3-diol, or a pharmaceutically acceptable salt, solvate or hydrate thereof in combination with another therapeutic agent.

Some embodiments describe a method for treating or preventing an AhR mediated disease selected from diseases that exhibit an inflammatory component, diseases that improve from inhibition of IL-17A and IL-17F expression, respiratory allergies, hay fever, skin allergies, chronic obstructive pulmonary disease (COPD), multiple sclerosis, systemic sclerosis, inflammatory bowel disease, irritable bowel disease (IBD), immune system dysregulation, aberrant cellular infiltrates, production of inflammatory mediators, deratinization, inflammation of the gut, Crohn's disease, ulcerative colitis, arthritis, rheumatoid arthritis, osteoarthritis, gluten-sensitive enteropathy (celiac disease), Hashimoto's thyroiditis, Sjogren's syndrome, Guillain-Barre syndrome, Grave's disease, Addison's disease (autoimmune disease of the adrenal glands), autoimmune polyglandular disease (autoimmune polyglandular syndrome), pernicious anemia, autoimmune hypopituitarism, glomerulonephritis, serum sickness, acute respiratory distress syndrome, acute inflammation, Behcet's disease, and Sezary syndrome, in a subject in need thereof, comprising administering to said subject an effective amount of a compound or pharmaceutical composition according to any embodiment described herein, or pharmaceutically acceptable salt, solvate or hydrate thereof. In some embodiments the compound is a compound of Formula (I) or Formula (Ia) or a pharmaceutically acceptable salt, solvate or hydrate thereof. In some embodiments the compound is a compound of Formula (I) or Formula (Ia) or pharmaceutically acceptable salt, solvate or hydrate thereof in combination with another therapeutic agent. In some embodiments the compound is 2-isopropyl-5-(isoquinolin-3-yl)benzene-1,3-diol, or a pharmaceutically acceptable salt, solvate or hydrate thereof. In some embodiments the compound is 2-isopropyl-5-(isoquinolin-3-yl)benzene-1,3-diol, or a pharmaceutically acceptable salt, solvate or hydrate thereof in combination with another therapeutic agent.

Some embodiments describe a method for treating or preventing an AhR mediated disease selected from inflammatory eye conditions, eye injuries, age-related macular degeneration, neovascular (dry) age-related macular degeneration, neovascular (wet) age-related macular degeneration, Fuchs endothelial corneal dystrophy, and uveitis, in a subject in need thereof, comprising administering to said subject an effective amount of a compound or pharmaceutical composition according to any embodiment described herein, or pharmaceutically acceptable salt, solvate or hydrate thereof. In some embodiments the compound is a compound of Formula (I) or Formula (Ia) or a pharmaceutically acceptable salt, solvate or hydrate thereof. In some embodiments the compound is a compound of Formula (I) or Formula (Ia) or pharmaceutically acceptable salt, solvate or hydrate thereof in combination with another therapeutic agent. In some embodiments the compound is 2-isopropyl-5-(isoquinolin-3-yl)benzene-1,3-diol, or a pharmaceutically acceptable salt, solvate or hydrate thereof. In some embodiments the compound is 2-isopropyl-5-(isoquinolin-3-yl)benzene-1,3-diol, or a pharmaceutically acceptable salt, solvate or hydrate thereof in combination with another therapeutic agent.

Vitiligo is a depigmentation disorder resulting from selective destruction of melanocytes. In melanocytes, AhR links solar UVB radiation to skin pigmentation. A decreased risk of vitiligo associated with a specific variant of the AhR gene has been described, and in further work, researchers found an AhR variant promoted Ahr transcriptional activity, facilitating its interaction with the SP1 transcripion factor, resulting in increased AhR expression and IL-10 production in humans.

Some embodiments herein describe a method of treating or preventing vitiligo in a subject in need thereof, the method comprising administering to said subject an effective amount of a compound or pharmaceutical composition according to any embodiment described herein, or pharmaceutically acceptable salt, solvate or hydrate thereof. In some embodiments the compound is a compound of Formula (I) or Formula (Ia) or a pharmaceutically acceptable salt, solvate or hydrate thereof. In some embodiments the compound is a compound of Formula (I) or Formula (Ia) or pharmaceutically acceptable salt, solvate or hydrate thereof in combination with another therapeutic agent. In some embodiments the compound is 2-isopropyl-5-(isoquinolin-3-yl)benzene-1,3-diol, or a pharmaceutically acceptable salt, solvate or hydrate thereof. In some embodiments the compound is 2-isopropyl-5-(isoquinolin-3-yl)benzene-1,3-diol, or a pharmaceutically acceptable salt, solvate or hydrate thereof in combination with another therapeutic agent.

Some embodiments describe a method of treating a disease as described above in a subject in need thereof, comprising administering to the subject, a therapeutically effective amount of 2-isopropyl-5-(isoquinolin-3-yl)benzene-1,3-diol:

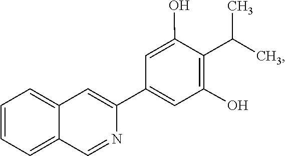 [SMILES: CC(C)c1c(O)cc(-c2cc3ccccc3cn2)cc1O]

or a pharmaceutically acceptable salt, solvate or hydrate thereof or a pharmaceutical composition thereof.

A compound according to any embodiment described herein may be used in a veterinary setting or in a medical setting. It is recognized that the subject or patient may be an animal, a domestic animal, such as a mammal, including horses, cows, pigs, sheep, poultry, fish, cats, dogs and zoo animals. In some embodiment, the subject is an animal.

In some embodiments, the subject is a mammal. In some embodiments, the subject is a human. In some embodiments, the human is an adult, or a pediatric patient. In some embodiments, the human is a pediatric patient. In some embodiments, the pediatric patient is a child. In some embodiments, the pediatric patient is 3 months to 2 years of age and older. In some embodiments, the human is an adult.

Some embodiments describe a compound according to any embodiment described herein, or pharmaceutically acceptable salt, solvate or hydrate thereof, for use in therapy. In some embodiments the compound is a compound of Formula (I) or Formula (Ia) or a pharmaceutically acceptable salt, solvate or hydrate thereof. In some embodiments the compound is 2-isopropyl-5-(isoquinolin-3-yl)benzene-1,3-diol, or a pharmaceutically acceptable salt, solvate or hydrate thereof.

Some embodiments describe a compound according to any embodiment described herein, or pharmaceutically acceptable salt, solvate or hydrate thereof, for use in the treatment or prevention of an AhR mediated disease. In some embodiments the compound is a compound of Formula (I) or Formula (Ia), or pharmaceutically acceptable salt, solvate or hydrate thereof. In some embodiments the compound is 2-isopropyl-5-(isoquinolin-3-yl)benzene-1,3-diol, or a pharmaceutically acceptable salt, solvate or hydrate thereof.

Some embodiments describe use of a compound according to any embodiment described herein, or a pharmaceutically acceptable salt, solvate or hydrate thereof, in the manufacture of a medicament for the treatment of an AhR mediated disease in a subject in need thereof. In some embodiments the compound is a compound of Formula (I) or Formula (Ia), or a pharmaceutically acceptable salt, solvate or hydrate thereof. In some embodiments the compound is 2-isopropyl-5-(isoquinolin-3-yl)benzene-1,3-diol, or a pharmaceutically acceptable salt, solvate or hydrate thereof.

Some embodiments describe use of a compound according to any embodiment described herein, or pharmaceutically acceptable salt, solvate or hydrate thereof, for the treatment or prevention of AhR mediated disease. In some embodiments the compound is a compound of Formula (I) or Formula (Ia), or pharmaceutically acceptable salt, solvate or hydrate thereof. In some embodiments the compound is 2-isopropyl-5-(isoquinolin-3-yl)benzene-1,3-diol, or a pharmaceutically acceptable salt, solvate or hydrate thereof.

### Combinations

For the pharmaceutical compositions and the methods/uses described herein, a compound of any embodiment described herein, may be administered in combination with one or more other therapies and/or active agents. In some embodiments, the compound of any embodiment described herein is administered in combination with a second agent indicated for the disorders or diseases described herein, either concomitant with, prior to, or after the administration of the second agent. In some embodiments the second agent is in the same formulation as a compound according to any embodiment described herein. In some embodiments, the second agent is in a separate formulation. The second therapeutic agent may be administered by the same route as the compound according to any embodiment described herein, or it may be administered by a different route than the compound according to any embodiment described herein. For example, a compound according to any embodiment described herein may be administered topically and the second agent may be administered topical, orally, intravenously intramuscularly, otic, opthamologicaly, opthalmically, vaginally, rectally, etc. In some embodiments the second agent is administered concomitant with a compound of the invention. In some embodiments the second agent is administered prior to the compound of the invention. In some embodiments the second agent is administered after the compound of the invention.

In other words, a compound according to any embodiment described herein, may be administered together, contemporaneously or sequentially in either order to the site of administration, or to a desired site of action. The order of administration is not deemed necessary. However, if topically administered it may be preferable that the two or more actives are in contact at some point together at the site of administration or desired site of action. Alternatively, it is desirable that the time period for appropriate mode of action of the actives is timed appropriately in the delivery time of the active. If both are present in the same vehicle they provide ease of administration to the patient, and perhaps increased compliance, but it is not required for the invention herein.

When a compound according to any embodiment described herein, is administered in combination with one or more other therapies and/or active agents as described herein, each of the active drug components (i.e., the compound of the present invention and the second agent) is contained in an effective dosage amount.

In some embodiments, the second agent is an agent for treating or preventing a condition associated with an AhR imbalance.

In some embodiments the other agent(s) is useful in the prevention or treatment of allergic disease, inflammatory disease, or autoimmune disease. In some embodiments the agent(s) is antigen immunotherapy agents; anti-histamines; corticosteroids, for example, fluticasone propionate, fluticasone furoate, beclomethasone dipropionate, budesonide, ciclesonide, mometasonefuroate, triamcinolone, and flunisolide; NSAIDs; leukotriene modulators, such as montelukast, zafirlukast, and pranlukast; iNOS inhibitors; tryptase inhibitors; IKK2 inhibitors; p38 inhibitors; Syk inhibitors; protease inhibitors; elastase inhibitors; integrin antagonists, for example, beta-2integrin antagonists; adenosine A2a agonists; mediator release inhibitors, for example, sodium chromoglycate, 5-lipoxygenase inhibitors (zyflo); DP1 antagonists; DP2 antagonists; PI3K delta inhibitors; ITK inhibitors; LP (lysophosphatidic) inhibitors; or FLAP (5-lipoxygenase activating protein) inhibitors, for example, sodium 3-(3-(tert-butylthio)-1-(4-(6-ethoxypyridin-3-yl)benzyl)-5-((5-methylpyridin-2-yl)methoxy)-1H-indol-2-yl)-2,2-dimethylpropanoate); bronchodilators, for example, muscarinic antagonists and beta-2 agonists; methotrexate, and similar agents; monoclonal antibody therapy agents such as anti-IgE, anti-TNF, anti-IL-5, anti-IL-6, anti-IL-12, anti-IL-1 and similar agents; cytokine receptor therapies, for examples, etanercept and similar agents; and antigen non-specific immunotherapies, for example, interferon or other cytokines/chemokines, chemokine receptor modulators such as CCR3, CCR4 or CXCR2 antagonists, other cytokine/chemokine agonists or antagonists, TLR agonists and similar agents.

In some embodiments the other agent(s) is an agent for aiding transplantation, including cyclosporines, tacrolimus, mycophenolate mofetil, prednisone, azathioprine, sirolimus, daclizumab, basiliximab, or OKT3.

In some embodiments the other agent(s) is an agent for treating diabetes, for example, metformin (biguanides), meglitinides, sulfonylureas, DPP-4 inhibitors, thiazolidinediones, or alpha-glucosidase inhibitors, amylin mimetics, incretin mimetics, or insulin.

In some embodiments the other agent(s) is an antihypertensive such as diuretics, ACE inhibitors, ARBS, calcium channel blockers, and beta blockers.

Accordingly, the disclosure provides, in a further aspect, a pharmaceutical composition comprising at least one compound according to any embodiment described herein, or pharmaceutically acceptable derivative thereof; a second active agent; and optionally, a pharmaceutically acceptable carrier.

When combined in the same formulation it will be appreciated that the two or more compounds must be stable and compatible with each other and the other components of the formulation. When formulated separately they may be provided in any convenient formulation, in such manner as are known for such compounds in the art.

Preservatives, stabilizers, dyes and flavoring agents may be provided in any pharmaceutical composition described herein. Examples of preservatives include sodium benzoate, ascorbic acid and esters of p-hydroxybenzoic acid. Antioxidants and suspending agents may be also used.

With respect to combinations including biologics such as monoclonal antibodies or fragments, suitable excipients will be employed to prevent aggregation and stabilize the antibody or fragment in solution with low endotoxin, generally for parenteral administration, for example, intravenous, administration. For example, see Formulation and Delivery Issues for Monoclonal Antibody Therapeutics, Daugherty et al., in Current Trends in Monoclonal Antibody Development and Manufacturing, Part 4, 2010, Springer, New York pp 103-129.

### Routes of Administration and Unit Dosage Forms

Compounds according to any embodiment described herein and pharmaceutical compositions incorporating said compounds may conveniently be administered by any of the routes conventionally used for drug administration, for instance, orally, topically, transdermally, parenterally or by inhalation. The compounds may be administered in conventional dosage forms prepared by combining a compound according to any embodiment described herein with standard pharmaceutical carriers according to conventional procedures. A compound according to any embodiment described herein, may also be administered in conventional dosages in combination with known, second therapeutically active compounds as further described herein. These procedures may involve mixing, granulating and compressing or dissolving the ingredients as appropriate to the desired preparation. It will be appreciated that the form and character of the pharmaceutically acceptable character or diluent is dictated by the amount of active ingredient with which it is to be combined, the route of administration and other well-known variables. The carrier(s) must be “acceptable” in the sense of being compatible with the other ingredients of the formulation and not deleterious to the recipient thereof.

A compound according to any embodiment described herein, may be administered topically, that is by non-systemic administration. This includes the application of the compound externally to the epidermis, the buccal cavity, or the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as liniments, lotions, creams, gels, solutions, ointments, pastes, and drops suitable for administration to the skin, the eye, ear or nose.

Lotions according to the present invention include those suitable for application to the skin, ear, nose or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, gels, ointments or pastes according to the present invention are semi-solid formulations of the active ingredient for external application. They may be made by mixing the active ingredient (i.e., a compound according to any embodiment described herein) in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenyl mercuric nitrate or acetate (0.002%), benzalkonium chloride (0.01%) and chlorhexidine acetate (0.01%). Suitable solvents for the preparation of an oily solution include glycerol, diluted alcohol and propylene glycol.

In some embodiments, a compound according to any embodiment described herein is administered topically in as a cream, gel ointment, paste, drop or lotion. In some embodiments a compound according to any embodiment described herein is administered as a gel or cream. In some embodiments a compound according to any embodiment described herein is administered as a gel. In some embodiments a compound according to any embodiment described herein is administered as a cream.

In some embodiments, the pharmaceutical formulation comprises a compound according to any embodiment described herein, and a pharmaceutically acceptable excipient or diluent and an anti-oxidant, preservative, gelling agent, pH adjusting agent, or stabilizer, or mixtures thereof suitably adapted for topical administration to the skin, eye, or ear of a patient.

In some embodiments the composition is a cream or gel composition and the compound of Formula (I) is 2-isopropyl-5-(isoquinolin-3-yl)benzene-1,3-diol.

In some embodiments the composition is a cream formulation. In some embodiments the composition is cream formulation 1 comprising the following:

| Formulation Component | Composition (% w/w) |
|---|---|
| 2-isopropyl-5-(isoquinolin-3-yl)benzene-1,3-diol | 0-1 |
| Propylene glycol | 15.0 |
| Transcutol P | 10.0 |
| PEG400 | 20.0 |
| Miglyol 810 | 5.0 |
| PEG40 Stearate | 1.0 |
| Glyceryl monostearate | 1.5 |
| Stearic acid | 0.15 |
| Cetostearyl alcohol 50 | 3.0 |
| Benzyl alcohol | 1.0 |
| BHT | 0.1 |
| Water | QS 100 |

QS 100: the amount needed to result in a composition totaling 100%.

It will be understood that when the 2-isopropyl-5-(isoquinolin-3-yl)benzene-1,3-diol in cream formulation 1 is present at a particular concentration (in the range of 0-1%), the formulation may be referred to as X % cream formulation 1. For example, in some embodiments the composition is a cream formulation 1, comprising 1% of 2-isopropyl-5-(isoquinolin-3-yl)benzene-1,3-diol (1% cream formulation 1). Thus, 1% cream formulation 1 comprises:

| Formulation Component | Composition (% w/w) |
|---|---|
| 2-isopropyl-5-(isoquinolin-3-yl)benzene-1,3-diol | 1 |
| Propylene glycol | 15.0 |
| Transcutol P | 10.0 |
| PEG400 | 20.0 |
| Miglyol 810 | 5.0 |
| PEG40 Stearate | 1.0 |
| Glyceryl monostearate | 1.5 |
| Stearic acid | 0.15 |
| Cetostearyl alcohol 50 | 3.0 |
| Benzyl alcohol | 1.0 |
| BHT | 0.1 |
| Water | QS 100 |

In some embodiments the composition is 0.5% cream formulation 1 comprising

| Formulation Component | Composition (% w/w) |
|---|---|
| 2-isopropyl-5-(isoquinolin-3-yl)benzene-1,3-diol | 0.5 |
| Propylene glycol | 15.0 |
| Transcutol P | 10.0 |
| PEG400 | 20.0 |
| Miglyol 810 | 5.0 |
| PEG40 Stearate | 1.0 |
| Glyceryl monostearate | 1.5 |
| Stearic acid | 0.15 |
| Cetostearyl alcohol 50 | 3.0 |
| Benzyl alcohol | 1.0 |
| BHT | 0.1 |
| Water | QS 100 |

In some embodiments the composition is 0.1% cream formulation 1 comprising

| Formulation Component | Composition (% w/w) |
|---|---|
| 2-isopropyl-5-(isoquinolin-3-yl)benzene-1,3-diol | 0.1 |
| Propylene glycol | 15.0 |
| Transcutol P | 10.0 |
| PEG400 | 20.0 |
| Miglyol 810 | 5.0 |
| PEG40 Stearate | 1.0 |
| Glyceryl monostearate | 1.5 |
| Stearic acid | 0.15 |
| Cetostearyl alcohol 50 | 3.0 |
| Benzyl alcohol | 1.0 |
| BHT | 0.1 |
| Water | QS 100 |

In some embodiments the composition is cream formulation 2 comprising the following:

| Formulation Component | Composition (% w/w) |
|---|---|
| 2-isopropyl-5-(isoquinolin-3-yl)benzene-1,3-diol | 0-1 |
| Propylene glycol | 15.0 |
| Transcutol P | 15.0 |
| PEG400 | 20.0 |
| Miglyol 810 | 5.0 |
| PEG40 Stearate | 1.0 |
| Glyceryl monostearate | 1.5 |
| Stearic acid | 0.15 |
| Cetostearyl alcohol 50 | 3.0 |
| Benzyl alcohol | 1.0 |
| BHT | 0.1 |
| Water | QS 100 |

In some embodiments the composition is cream formulation 3 comprising the following:

| Formulation Component | Composition (% w/w) |
|---|---|
| 2-isopropyl-5-(isoquinolin-3-yl)benzene-1,3-diol | 0-1 |
| Propylene glycol | 35.0 |
| Transcutol P | 15.0 |
| Miglyol 810 | 5.0 |
| Steareth 21 | 5 |
| Steareth 2 | |
| Cetostearyl alcohol 50 | 3.0 |
| Benzyl alcohol | 1.0 |
| BHT | 0.1 |
| Water | QS 100 |

In some embodiments the composition is 1% cream formulation 3 comprising

| Formulation Component | Composition (% w/w) |
|---|---|
| 2-isopropyl-5-(isoquinolin-3-yl)benzene-1,3-diol | 1 |
| Propylene glycol | 35.0 |
| Transcutol P | 15.0 |
| Miglyol 810 | 5.0 |
| Steareth 21 | 5 |
| Steareth 2 | |
| Cetostearyl alcohol 50 | 3.0 |
| Benzyl alcohol | 1.0 |
| BHT | 0.1 |
| Water | QS 100 |

In some embodiments the composition is 0.5% cream formulation 3 comprising

| Formulation Component | Composition (% w/w) |
|---|---|
| 2-isopropyl-5-(isoquinolin-3-yl)benzene-1,3-diol | 0.5 |
| Propylene glycol | 35.0 |
| Transcutol P | 15.0 |
| Miglyol 810 | 5.0 |
| Steareth 21 | 5 |
| Steareth 2 | |
| Cetostearyl alcohol 50 | 3.0 |
| Benzyl alcohol | 1.0 |
| BHT | 0.1 |
| Water | QS 100 |

In some embodiments the composition is cream formulation 4 comprising the following:

| Formulation Component | Composition (% w/w) |
|---|---|
| 2-isopropyl-5-(isoquinolin-3-yl)benzene-1,3-diol | 0-1 |
| Propylene glycol | 15.0 |
| Transcutol P | 25.0 |
| Miglyol 810 | 5.0 |
| Steareth 21 | 5 |
| Steareth 2 | |
| Cetostearyl alcohol 50 | 3.0 |
| Benzyl alcohol | 1.0 |
| BHT | 0.1 |
| Carbomer p980 | 0.25 |
| Triethanolamine | 0.2 |
| Water | QS 100 |

In some embodiments the composition is cream formulation 5 comprising the following:

| Formulation Component | Composition (% w/w) |
|---|---|
| 2-isopropyl-5-(isoquinolin-3-yl)benzene-1,3-diol | 0-1 |
| Propylene glycol | 15.0 |
| Transcutol P | 25.0 |
| Miglyol 810 | 5.0 |
| Steareth 21 | 5 |
| Steareth 2 | |
| Cetostearyl alcohol 50 | 3.0 |
| Benzyl alcohol | 1.0 |
| BHT | 0.1 |
| Xanthan gum | 0.75 |
| Water | QS 100 |

In some embodiments the composition is cream formulation 6 comprising the following:

| Formulation Component | Composition (% w/w) |
|---|---|
| 2-isopropyl-5-(isoquinolin-3-yl)benzene-1,3-diol | 0-1 |
| Propylene glycol | 15.0 |
| Transcutol P | 25.0 |
| Miglyol 810 | 5.0 |
| Steareth 21 | 5 |
| Steareth 2 | |
| Cetostearyl alcohol 50 | 3.0 |
| Benzyl alcohol | 1.0 |
| BHT | 0.1 |
| Xanthan gum | 0.15 |
| Water | QS 100 |

In some embodiments the composition is cream formulation 7 comprising the following:

| Formulation Component | Composition (% w/w) |
|---|---|
| 2-isopropyl-5-(isoquinolin-3-yl)benzene-1,3-diol | 0-1 |
| Propylene glycol | 15.0 |
| Transcutol P | 25.0 |
| Miglyol 810 | 5.0 |
| Steareth 21 | 5 |
| Steareth 2 | |
| Cetostearyl alcohol 50 | 3.0 |
| Benzyl alcohol | 1.0 |
| BHT | 0.1 |
| Water | QS 100 |

In some embodiments the composition is a gel formulation (gel formulation 1) comprising the following:

| Formulation Component | Composition (% w/w) |
|---|---|
| 2-isopropyl-5-(isoquinolin-3-yl)benzene-1,3-diol | 0-1 |
| Propylene glycol | 10.0 |
| Transcutol P | 35.0 |
| Benzyl alcohol | 2.0 |
| BHT | 0.05 |
| Carbomer p980 | 0.5 |
| Trolamine | 0.15 |
| Water | QS 100 |

In some embodiments the composition is a gel formulation (1% gel formulation 1) comprising the following:

| Formulation Component | Composition (% w/w) |
|---|---|
| 2-isopropyl-5-(isoquinolin-3-yl)benzene-1,3-diol | 1 |
| Propylene glycol | 10.0 |
| Transcutol P | 35.0 |
| Benzyl alcohol | 2.0 |
| BHT | 0.05 |
| Carbomer p980 | 0.5 |
| Trolamine | 0.15 |
| Water | QS 100 |

In some embodiments the composition is a gel formulation (gel formulation 2) comprising the following:

| Formulation Component | Composition (% w/w) |
|---|---|
| 2-isopropyl-5-(isoquinolin-3-yl)benzene-1,3-diol | 0-1 |
| Transcutol P | 35.0 |
| PEG400 | 10.0 |
| Benzyl alcohol | 2.0 |
| BHT | 0.05 |
| Carbomer p980 | 0.5 |
| Trolamine | 0.2 |
| Water | QS 100 |

In some embodiments the composition is a gel formulation (gel formulation 3) comprising the following:

| Formulation Component | Composition (% w/w) |
|---|---|
| 2-isopropyl-5-(isoquinolin-3-yl)benzene-1,3-diol | 0-1 |
| Propylene glycol | 20.0 |
| Transcutol P | 25.0 |
| Benzyl alcohol | 2.0 |
| BHT | 0.05 |
| Carbomer p980 | 0.5 |
| Trolamine | 0.15 |
| Water | QS 100 |

In some embodiments the composition is a gel formulation (gel formulation 4) comprising the following:

| Formulation Component | Composition (% w/w) |
|---|---|
| 2-isopropyl-5-(isoquinolin-3-yl)benzene-1,3-diol | 0-1 |
| Propylene glycol | 40.0 |
| Transcutol P | 15.0 |
| Benzyl alcohol | 2.0 |
| BHT | 0.05 |
| Carbomer p980 | 0.5 |
| Trolamine | 0.15 |
| Water | QS 100 |

While topical usage is a preferred administration route, a compound according to any embodiment described herein, may also be administered parenterally, that is by intravenous, intramuscular, subcutaneous, intranasal, intrarectal, intravaginal or intraperitoneal administration. The subcutaneous and intramuscular forms of parenteral administration are generally preferred. Appropriate dosage forms for such administration may be prepared by conventional techniques. A compound according to any embodiment described herein, may also be administered by inhalation, that is by intranasal and oral inhalation administration. Appropriate dosage forms for such administration, such as an aerosol formulation or a metered dose inhaler, may be prepared by conventional techniques.

The dosage of a compound according to any embodiment described herein, as an active ingredient of this invention may be varied so that a suitable dosage form is obtained. The active ingredient may be administered to subjects (animals and human) in need of such treatment in dosages that will provide optimal pharmaceutical efficacy. The selected dosage depends upon the desired therapeutic effect, on the route of administration, and on the duration of the treatment. The dose will vary from patient to patient depending upon the nature and severity of disease, the patient's weight, special diets then being followed by a patient, concurrent medication, and other factors which those skilled in the art will recognize.

In some embodiments, the amount of the compound to be administered can range between about 0.1 and about 100 mg/kg/day. Generally, dosage levels of between 0.1 to 10 mg/kg of body weight daily are administered to the patient, e.g., humans. In some embodiments the therapeutically effective amount is between a lower limit of about 0.1 mg/kg of body weight, about 0.2 mg/kg of body weight, about 0.3 mg/kg of body weight, about 0.4 mg/kg of body weight, about 0.5 mg/kg of body weight, about 0.6 mg/kg of body weight, about 0.7 mg/kg of body weight, about 0.8 mg/kg of body weight, about 0.9 mg/kg of body weight, about 1 mg/kg of body weight, about 5 mg/kg of body weight, about 10 mg/kg of body weight, about 15 mg/kg of body weight, about 20 mg/kg of body weight, about 25 mg/kg of body weight, about 30 mg/kg of body weight, about 35 mg/kg of body weight, about 40 mg/kg of body weight, about 45 mg/kg of body weight, about 50 mg/kg of body weight, 55 mg/kg of body weight, about 60 mg/kg of body weight, about 65 mg/kg of body weight, about 70 mg/kg of body weight, about 75 mg/kg of body weight, about 80 mg/kg of body weight, about 85 mg/kg of body weight, about 80 mg/kg of body weight, about 95 mg/kg of body weight, and about 100 mg/kg of body weight; and an upper limit of 100 mg/kg of body weight, about 95 mg/kg of body weight, about 90 mg/kg of body weight, about 85 mg/kg of body weight, about 80 mg/kg of body weight, about 75 mg/kg of body weight, about 70 mg/kg of body weight, about 65 mg/kg of body weight, about 60 mg/kg of body weight, about 55 mg/kg of body weight 50 mg/kg of body weight, about 45 mg/kg of body weight, about 40 mg/kg of body weight, about 35 mg/kg of body weight, about 30 mg/kg of body weight, about 25 mg/kg of body weight, about 20 mg/kg of body weight, about 15 mg/kg of body weight, about 10 mg/kg of body weight, about 5 mg/kg of body weight, about 1 mg/kg of body weight, about 0.9 mg/kg of body weight, about 0.8 mg/kg of body weight, about 0.7 mg/kg of body weight, about 0.6 mg/kg of body weight, about 0.5 mg/kg of body weight, about 0.4 mg/kg of body weight, about 0.3 mg/kg of body weight, about 0.2 mg/kg of body weight, and about 0.1 mg/kg of body weight per day.

In some embodiments, a compound according to any embodiment described herein is administered to a subject at a total daily dose of about 0.01 to about 1000 mg/day. In some embodiments the total daily dose is about 0.1 to about 100 mg/day. In some embodiments the total daily dose is between a lower limit of about 0.01 mg/day, about 0.05 mg/day, 0.1 mg/day, about 0.5 mg/day, about 1 mg/day, about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, about 500 mg/day, about 510 mg/day, about 520 mg/day, about 530 mg/day, about 540 mg/day, about 550 mg/day, about 560 mg/day, about 570 mg/day, about 580 mg/day, about 590 mg/day, about 600 mg/day, about 610 mg/day, about 620 mg/day, about 630 mg/day, about 640 mg/day, about 650 mg/day, about 660 mg/day, about 670 mg/day, about 680 mg/day, about 690 mg/day, about 700 mg/day, about 710 mg/day, about 720 mg/day, about 730 mg/day, about 740 mg/day, about 750 mg/day, about 760 mg/day, about 770 mg/day, about 780 mg/day, about 790 mg/day, about 800 mg/day, about 810 mg/day, about 820 mg/day, about 830 mg/day, about 840 mg/day, about 850 mg/day, about 860 mg/day, about 870 mg/day, about 880 mg/day, about 890 mg/day, about 900 mg/day, about 910 mg/day, about 920 mg/day, about 930 mg/day, about 940 mg/day, about 950 mg/day, about 960 mg/day, about 970 mg/day, about 980 mg/day, about 990 mg/day, and about 1000 mg/day, and an upper limit of 1000 mg/day, about 990 mg/day, about 980 mg/day, about 970 mg/day, about 960 mg/day, about 950 mg/day, about 940 mg/day, about 930 mg/day, about 920 mg/day, about 910 mg/day, about 900 mg/day, about 890 mg/day, about 880 mg/day, about 870 mg/day, about 860 mg/day, about 850 mg/day, about 840 mg/day, about 830 mg/day, about 820 mg/day, about 810 mg/day, about 800 mg/day, about 790 mg/day, about 780 mg/day, about 770 mg/day, about 760 mg/day, about 750 mg/day, about 740 mg/day, about 730 mg/day, about 720 mg/day, about 710 mg/day, about 700 mg/day, about 690 mg/day, about 680 mg/day, about 670 mg/day, about 660 mg/day, about 650 mg/day, about 640 mg/day, about 630 mg/day, about 620 mg/day, about 610 mg/day, about 600 mg/day, about 590 mg/day, about 580 mg/day, about 570 mg/day, about 560 mg/day, about 550 mg/day, about 540 mg/day, about 530 mg/day, about 520 mg/day, about 510 mg/day, about 500 mg/day, about 490 mg/day, about 480 mg/day, about 470 mg/day, about 460 mg/day, about 450 mg/day, about 440 mg/day, about 430 mg/day, about 420 mg/day, about 410 mg/day, about 400 mg/day, about 390 mg/day, about 380 mg/day, about 370 mg/day, about 360 mg/day, about 350 mg/day, about 340 mg/day, about 330 mg/day, about 320 mg/day, about 310 mg/day, about 300 mg/day, about 290 mg/day, about 280 mg/day, about 270 mg/day, about 260 mg/day, about 250 mg/day, about 240 mg/day, about 230 mg/day, about 220 mg/day, about 210 mg/day, about 200 mg/day, about 190 mg/day, about 180 mg/day, about 170 mg/day, about 160 mg/day, about 150 mg/day, about 140 mg/day, about 130 mg/day, about 120 mg/day, about 110 mg/day, about 100 mg/day, about 90 mg/day, about 80 mg/day, about 70 mg/day, about 60 mg/day, about 50 mg/day, about 40 mg/day, about 30 mg/day, about 10 mg/day, about 1 mg/day, about 0.5 mg/day, about 0.1 mg/day, and about 0.01 mg/day.

It will be understood that the pharmaceutical compositions of the disclosure need not necessarily contain the entire amount of the compound that is effective in treating the disorder, as such effective amounts can be reached by administration of a plurality of divided doses of such pharmaceutical compositions. The compounds may be administered in a single dose per day or on a regimen of multiple doses per day, (e.g., two, three, four, five, or more) of sub-doses per day such that the total daily dose is the same. An effective amount of a salt thereof may be determined as a proportion of the effective amount of the compound of according to any embodiment described herein. Similar dosages should be appropriate for treatment of the other conditions referred herein for treatment. In general, determination of appropriate dosing can be readily arrived at by one skilled in medicine or the pharmaceutical arts.

The active ingredient, i.e., a compound according to any embodiment described herein, may be for topical administration administered from about 0.001% w/w to about 10% w/w of the topical formulation. In some embodiments, a compound according to any embodiment described herein is 0.1% w/w, 0.2% w/w, 0.3% w/w, 0.4% w/w, 0.5% w/w, 0.6% w/w, 0.7% w/w, 0.8% w/w, 0.9% w/w, 1% w/w, 2% w/w, 3% w/w, 4% w/w, 5% w/w, 6% w/w, 7% w/w, 8% w/w, 9% w/w, or 10% w/w of the topical formulation. In some embodiments a compound according to any embodiment described herein is from 1% w/w to 2% w/w of the formulation. The daily topical dosage regimen may be from about 0.1 mg to 150 mg of a compound according to any embodiment described herein, administered one to four times daily. In some embodiments the daily topical dose is 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, or 150 mg. Initial dosages can also be estimated from in vivo data, using animal models. Animal models useful for testing the efficacy of compounds to treat or prevent the various diseases described above are well-known in the art. The compounds may be administered once per week, several times per week (for example, every other day), once per day or multiple times per day, depending upon the judgment of the prescribing physician.

It will also be recognized by one of skill in the art that the optimal quantity and spacing of individual dosages of a compound according to any embodiment described herein, or a pharmaceutically acceptable salt, solvate or hydrate thereof will be determined by the nature and extent of the condition being treated, the form, route and site of administration, and the patient being treated, and that such optimums can be determined by conventional techniques. It will also be appreciated by one of skill in the art that the optimal course of treatment, i.e., the number of doses of a compound according to any embodiment described herein or a pharmaceutically acceptable salt, solvate or hydrate thereof, given per day for a defined number of days, can be ascertained by those skilled in the art using conventional course of treatment determination tests.

The compounds described herein may be made by processes disclosed in U.S. Pat. No. 11,267,788 and WO 2022/015423.

Appropriate amounts in any given instance will be readily apparent to those skilled in the art or capable of determination by routine experimentation. The compositions are generally applied in topical manner to the affected area, i. e., localized application to the skin region where the clinical abnormality is manifest.

Unless otherwise indicated, all percentages are based on the percent by weight of the final composition prepared, and all totals equal 100% by weight.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative and not a limitation of the scope of the present invention in any way. The embodiments of the invention in which an exclusive property or privilege is claimed are defined as follows.

What is claimed is:

1. A method of treating a disease in a subject in need thereof, comprising administering to said subject a therapeutically effective amount of a compound of Formula (I) or Formula (Ia) as described herein, or a pharmaceutically acceptable salt, solvate or hydrate thereof, wherein the disease is selected from the group consisting of Pemphigus foliaceus, Pemphigus vulgaris, paraneoplastic pemphigus, bullous pemphigoid, epidermolysis bullosa acquisita, lupus erythematosus, dermatomyositis, myasthenia gravis, goodpadture's syndrome, and autoimmune thyroiditis.

2. A method of treating a disease in a subject in need thereof, comprising administering to said subject a therapeutically effective amount of a compound of Formula (I) or Formula (Ia) as described herein, or a pharmaceutically acceptable salt, solvate or hydrate thereof, wherein the disease is selected from the group consisting of Sweet's syndrome, pustular psoriasis, pyoderma gangrenosum, hidradenitis suppuritiva, leukocytoclastic vasculitis Wegener's Granulomatosis, and neutrophilic dermatoses.

3. A method of treating a disease in a subject in need thereof, comprising administering to said subject a therapeutically effective amount of a compound of Formula (I) or Formula (Ia) as described herein, or a pharmaceutically acceptable salt, solvate or hydrate thereof, wherein the disease is selected from the group consisting of allergic diseases, autoimmune diseases, inflammatory airway disease, allergic rhinitis, autoimmune blistering diseases, Pemphigus vulgaris and Pemphigus foliaceus, bullous pemphigoid, inflammatory joint disease, Schamberg's disease, pigmented purpuric dermatoses (PPDs), lupus vulgaris, and dermatomyositis.

4. A method of treating a disease in a subject in need thereof, comprising administering to said subject a therapeutically effective amount of a compound of Formula (I) or Formula (Ia) as described herein, or a pharmaceutically acceptable salt, solvate or hydrate thereof, wherein the disease is selected from the group consisting of mastocytosis, urticaria, rosacea, fibromyalgia, chronic pain syndromes, chronic pruritis, chronic itch, and allergic rhinitis.

5. A method of treating a disease in a subject in need thereof, comprising administering to said subject a therapeutically effective amount of a compound of Formula (I) or Formula (Ia) as described herein, or a pharmaceutically acceptable salt, solvate or hydrate thereof, wherein the disease is selected from the group consisting of arteriosclerosis, benign tumor growth, and malignant tumor growth.

6. A method of treating a disease in a subject in need thereof, comprising administering to said subject a therapeutically effective amount of a compound of Formula (I) or Formula (Ia) as described herein, or a pharmaceutically acceptable salt, solvate or hydrate thereof, wherein the disease is selected from the group consisting of inflammatory airway disease, allergic rhinitis, Schamberg's disease, pigmented purpuric dermatoses (PPDs), and inflammatory joint disease.

7. The method of any one of claims 1-6, wherein the compound is 2-isopropyl-5-(isoquinolin-3-yl)benzene-1,3-diol of the formula: 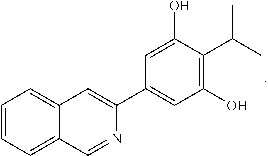 [SMILES: CC(C)c1c(O)cc(-c2cc3ccccc3cn2)cc1O]
